(19) United States
(12) Patent Application Publication
(10) Pub. No.: US 2026/0345601 A1
(43) Pub. Date: Nov. 5, 2026

(54) FORMS OF CARBAMATE DERIVATIVES AND RELATED USES

(71) Applicant: NodThera Limited, Little Chesterford, Saffron Walden Essex (GB)

(72) Inventor: Valérie READER, Linton, Cambridgeshire (GB)

(21) Appl. No.: 19/118,258

(22) PCT Filed: Oct. 4, 2023
(86) PCT No.: PCT/EP2023/077411

§ 371 Date: Apr. 3, 2025

Related U.S. Application Data
(60) Provisional application No. 63/413,036, filed on Oct. 4, 2022.

(51) Int. Cl. A61K 31/505; C07D 239/26

(52) U.S. Cl. CPC A61K 31/505; C07D 239/26

(57) ABSTRACT

The present disclosure relates to morphic forms of Compound A:

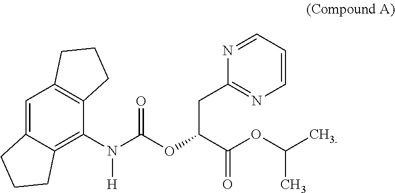 [SMILES: CC(C)OC(=O)[C@@H](Cc1ncccn1)OC(=O)Nc1c2c(cc3c1CCC3)CCC2]

The present disclosure also relates to processes for the preparation of the morphic forms of Compound A, the pharmaceutical compositions comprising the morphic forms, and the use thereof, e.g., in the treatment of disorders (e.g., in which inflammasome activity is implicated).

### RELATED APPLICATION

This application claims priority to U.S. Provisional Patent Application No. 63/413,036, filed Oct. 4, 2022, the entire content of which is hereby incorporated by reference.

### BACKGROUND

Autoimmune diseases are associated with the overproduction of proinflammatory factors. One of them is interleukin-1 (IL-1), produced by activated macrophages, monocytes, fibroblasts, and other components of the innate immune system like dendritic cells. IL-1 is involved in a variety of cellular activities, including cell proliferation, differentiation and apoptosis (Masters, S. L., et. al., Annu. Rev. Immunol. 2009. 27:621-68).

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation.

Although inflammasome activation appears to have evolved as an important component of host immunity to pathogens, the NLRP3 inflammasome is unique in its ability activate in response to endogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1 and IL-18 being implicated in a diverse range of pathophysiological indications including metabolic, physiologic, inflammatory, hematologic and immunologic disorders.

The disclosure arises from a need to provide further compounds for the specific modulation of NLRP3-dependent cellular processes. In particular, compounds with improved physicochemical, pharmacological and pharmaceutical properties to existing compounds are desirable.

### SUMMARY

In some aspects, the present disclosure provides a morphic form of Compound A.

In some aspects, the present disclosure provides a method of preparing a crystalline form of Compound A described herein.

In some aspects, the present disclosure provides a pharmaceutical composition comprising a morphic form of Compound A as described herein, and one or more pharmaceutically acceptable carriers or excipients.

In some aspects, the present disclosure provides a method of inhibiting inflammasome (e.g., the NLRP3 inflammasome) activity (e.g., in vitro or in vivo), comprising contacting a cell with an effective amount of a morphic form of Compound A.

In some aspects, the present disclosure provides a method of treating or preventing a disease or disorder disclosed herein in a subject in need thereof, comprising administering to the subject an effective amount of a morphic form of Compound A.

In some aspects, the present disclosure provides a morphic form of Compound A for use in inhibiting inflammasome (e.g., the NLRP3 inflammasome) activity (e.g., in vitro or in vivo).

In some aspects, the present disclosure provides a morphic form of Compound A for use in treating or preventing a disease or disorder disclosed herein.

In some aspects, the present disclosure provides use of a morphic form of Compound A in the manufacture of a medicament for inhibiting inflammasome (e.g., the NLRP3 inflammasome) activity (e.g., in vitro or in vivo).

In some aspects, the present disclosure provides use of a morphic form of Compound A in the manufacture of a medicament for treating or preventing a disease or disorder disclosed herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a graph showing the 1H-NMR of Form 1. The 1H-NMR spectrum was collected using the conditions described in the examples.

FIG. 2 is a graph showing the XRPD of Form 1.

FIG. 3 is a graph showing the TGA of Form 1.

FIGS. 4A and 4B are a set of photographs showing the SEM images of Form 1.

FIG. 5 is a graph showing the Raman spectrum of Form 1.

FIGS. 6A and 6B are a set of graphs showing the GVS Isothermal Plot and Kinetic Plot of Form 1.

FIG. 7 is a graph showing the ¹H-NMR of Form 2. The ¹H-NMR spectrum was collected using the conditions described in the examples.

FIG. 8 is a graph showing the XRPD of Form 2.

FIG. 9 is a graph showing the TGA of Form 2.

FIGS. 10A-10C are a set of photographs showing the PLM images of Form 2.

FIGS. 11A and 11B are a set of graphs showing the GVS Isothermal Plot and Kinetic Plot of Form 2.

### DETAILED DESCRIPTION

Autoimmune diseases are associated with the overproduction of proinflammatory factors. One of them is interleukin-1 (IL-1), produced by activated macrophages, monocytes, fibroblasts, and other components of the innate immune system like dendritic cells, involved in a variety of cellular activities, including cell proliferation, differentiation, and apoptosis (Masters, S. L., et al. Annu. Rev. Immunol. 2009. 27:621-68).

Cytokines from the IL-1 family are highly active and, as important mediators of inflammation, primarily associated with acute and chronic inflammation (Sims, J. et al., Nature Reviews Immunology 10, 89-102 (February 2010)). The overproduction of IL-1 is considered to be a mediator of some autoimmune and autoinflammatory diseases. Autoinflammatory diseases are characterised by recurrent and unprovoked inflammation in the absence of autoantibodies, infection, or antigen-specific T lymphocytes.

Proinflammatory cytokines of the IL-1 superfamily include IL-1α, IL-1β, IL-18, and IL-36α, β, λ and are produced in response to pathogens and other cellular stressors as part of a host innate immune response. Unlike many other secreted cytokines, which are processed and released via the standard cellular secretory apparatus consisting of the endoplasmic reticulum and Golgi apparatus, IL-1 family members lack leader sequences required for endoplasmic reticulum entry and thus are retained intracellularly following translation. In addition, IL-1β, IL-18, and IL-36α, β, λ are synthesised as procytokines that require proteolytic activation to become optimal ligands for binding to their cognate receptors on target cells.

In the case of IL-1α, IL-1β and IL-18, it is now appreciated that a multimeric protein complex known as an inflammasome is responsible for activating the proforms of IL-1β and IL-18 and for release of these cytokines extracellularly. An inflammasome complex typically consists of a sensor molecule, such as an NLR (Nucleotide-Oligerimisation Domain (NOD)-like receptor), an adaptor molecule ASC (Apoptosis-associated speck-like protein containing a CARD (Caspase Recruitment Domain)) and procaspase-1. In response to a variety of “danger signals”, including pathogen-associated molecule patterns (PAMPs) and danger associated molecular patterns (DAMPs), subunits of an inflammasome oligomerise to form a supramolecular structure within the cell. PAMPs include molecules such as peptidoglycan, viral DNA or RNA and bacterial DNA or RNA. DAMPs, on the other hand, consist of a wide range of endogenous or exogenous sterile triggers including monosodium urate crystals, silica, alum, asbestos, fatty acids, ceramides, cholesterol crystals and aggregates of beta-amyloid peptide. Assembly of an inflammasome platform facilitates autocatalysis of procaspase-1 yielding a highly active cysteine protease responsible for activation and release of pro-IL-1β and pro-IL-18. Thus, release of these highly inflammatory cytokines is achieved only in response to inflammasome sensors detecting and responding to specific molecular danger signals.

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation including NLRP1, NLRP3, NLRP6, NLRP7, NLRP12 and NLRC4 (IPAF).

Two other structurally distinct inflammasome structures containing a PYHIN domain (pyrin and HIN domain containing protein) namely Absent in Melanoma 2 (AIM2) and IFNλ-inducible protein 16 (IFI16) (Latz et al., Nat Rev Immunol 2013 13 (6) 397-311) serve as intracellular DNA sensors. Pyrin (encoded by the MEFV gene) represents another type of inflammasome platform associated with proIL-1β activation (Chae et al., Immunity 34, 755-768, 2011).

Requiring assembly of an inflammasome platform to achieve activation and release of IL-1β and IL-18 from monocytes and macrophages ensures their production is carefully orchestrated via a 2-step process. First, the cell must encounter a priming ligand (such as the TLR4 receptor ligand LPS, or an inflammatory cytokine such as TNFα) which leads to NFkB dependent transcription of NLRP3, pro-IL-1β and pro-IL-18. The newly translated procytokines remain intracellular and inactive unless producing cells encounter a second signal leading to activation of an inflammasome scaffold and maturation of procaspase-1.

In addition to proteolytic activation of pro-IL-1β and pro-IL-18, active caspase-1 also triggers a form of inflammatory cell death known as pyroptosis through cleavage of gasdermin-D. Pyroptosis allows the mature forms of IL-1β and IL-18 to be externalised along with release of alarmin molecules (compounds that promote inflammation and activate innate and adaptive immunity) such as high mobility group box 1 protein (HMGB1), IL-33, and IL-1α.

Although inflammasome activation appears to have evolved as an important component of host immunity to pathogens, the NLRP3 inflammasome is unique in its ability activate in response to endogenous and exogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1β and IL-18 being implicated in a diverse range of pathophysiological indications including metabolic, physiologic, inflammatory, hematologic and immunologic disorders.

A link to human disease is best exemplified by discovery that mutations in the NLRP3 gene which lead to gain-of-function confer a range of autoinflammatory conditions collectively known as cryopyrin-associated periodic syndromes (CAPS) including familial cold autoinflammatory syndrome (FCAS), Muckle-Wells syndrome (MWS) and Neonatal onset multisystem inflammatory disease (NOMID) (Hoffman et al., Nat. Genet. 29 (3) (2001) 301-305). Likewise, sterile mediator-induced activation of NLRP3 has been implicated in a wide range of disorders including joint degeneration (gout, rheumatoid arthritis, osteoarthritis), cardiometabolic (type 2 diabetes, atherosclerosis, hypertension), Central Nervous System (Alzheimer's Disease, Parkinson's disease, multiple sclerosis), gastrointestinal (Crohn's disease, ulcerative colitis), lung (chronic obstructive pulmonary disease (COPD), asthma, idiopathic pulmonary fibrosis) and liver (fibrosis, non-alcoholic fatty liver disease, non-alcoholic steatohepatitis (NASH)). It is further believed that NLRP3 activation promotes kidney inflammation and thus contributes to chronic kidney disease (CKD).

Current treatment options for diseases where IL-1 is implicated as a contributor to pathogenesis include the IL-1 receptor antagonist anakinra, an Fc-containing fusion construct of the extracellular domains of the IL-1 receptor and IL-1 receptor accessory protein (rilonacept) and the anti-IL-1β monoclonal antibody canakinumab. For example, canakinumab is licensed for CAPS, Tumor Necrosis Factor Receptor Associated Periodic Syndrome (TRAPS), Hyperimmunoglobulin D Syndrome (HIDS)/Mevalonate Kinase Deficiency (MKD), Familial Mediterranean Fever (FMF) and gout.

Some small molecules have been reported to inhibit function of the NLRP3 inflammasome. Glyburide, for example, is a specific inhibitor of NLRP3 activation, albeit at micromolar concentrations which are unlikely attainable in vivo. Non-specific agents such as parthenolide, Bay 11-7082, and 3,4-methylenedioxy-β-nitrostyrene are reported to impair NLRP3 activation but are expected to possess limited therapeutic utility due to their sharing of a common structural feature consisting of an olefin activated by substitution with an electron withdrawing group; this can lead to undesirable formation of covalent adducts with protein-bearing thiol groups. A number of natural products, for example β-hydroxybutyrate, sulforaphane, quercetin, and salvianolic acid, also are reported to suppress NLRP3 activation. Likewise, numerous effectors/modulators of other molecular targets have been reported to impair NLRP3 activation including agonists of the G-protein coupled receptor TGR5, an inhibitor of sodium-glucose co-transport epigliflozin, the dopamine receptor antagonist A-68930, the serotonin reuptake inhibitor fluoxetine, fenamate non-steroidal anti-inflammatory drugs, and the β-adrenergic receptor blocker nebivolol. Utility of these molecules as therapeutics for the chronic treatment of NLRP3-dependent inflammatory disorders remains to be established.

The disclosure relates to compounds useful for the specific modulation of NLRP3-dependent cellular processes. In particular, compounds with improved physicochemical, pharmacological and pharmaceutical properties to existing NLRP3-modulating compounds are desired.

### Morphic Forms of the Present Disclosure

It is understood that “Compound A” as used herein, refers to a compound with the structure shown below:

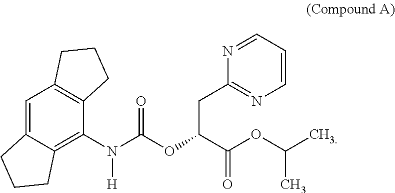 [SMILES: CC(C)OC(=O)[C@@H](Cc1ncccn1)OC(=O)Nc1c2c(cc3c1CCC3)CCC2]

It is understood that the chemical name of Compound A is propan-2-yl(2R)-2-{[(1,2,3,5,6,7-hexahydro-s-indacen-4-yl) carbamoyl]oxy}-3-(pyrimidin-2-yl) propanoate. It is understood that Compound A may be prepared as described in Example 101 in WO 2019/025467 (incorporated herein by reference).

In some aspects, the present disclosure provides a morphic form of Compound A.

In some embodiments, the morphic form is a crystalline form of Compound A.

In some embodiments, the morphic form is Form 1 of Compound A.

In some embodiments, the morphic form is Form 2 of Compound A.

### Form 1

In some embodiments, the morphic form of Compound A is Form 1 as described herein.

### X-Ray Powder Diffraction (XRPD) Characterizations

In some embodiments, the morphic form (e.g., Form 1) of Compound A is characterized by an XRPD pattern comprising signals (e.g., peaks) at 8.7±0.5, 15.3±0.5, and 15.9±0.5° 2θ (e.g., 8.7±0.2, 15.3±0.2, and 15.9±0.2° 2θ using Cu Kα radiation (e.g., 8.7±0.1, 15.3±0.1, and 15.9±0.1° 2θ using Cu Kα radiation (e.g., 8.7, 15.3, and 15.9° 2θ using Cu Kα radiation))).

In some embodiments, the morphic form (e.g., Form 1) of Compound A is characterized by an XRPD pattern comprising signals (e.g., peaks) at 7.9±0.5, 8.7±0.5, 15.3±0.5, and 15.9±0.5° 2θ (e.g., 7.9±0.2, 8.7±0.2, 15.3±0.2, and 15.9±0.2° 2θ using Cu Kα radiation (e.g., 7.9±0.1, 8.7±0.1, 15.3±0.1, and 15.9±0.1° 2θ using Cu Kα radiation (e.g., 7.9, 8.7, 15.3, and 15.9 °20 using Cu Kα radiation))).

In some embodiments, the morphic form (e.g., Form 1) of Compound A is characterized by an XRPD pattern comprising signals (e.g., peaks) at 7.9±0.5, 8.7±0.5, 15.3±0.5, 15.9±0.5, and 24.4±0.5° 2θ (e.g., 7.9±0.2, 8.7±0.2, 15.3±0.2, 15.9±0.2, and 24.4±0.2° 2θ using Cu Kα radiation (e.g., 7.9±0.1, 8.7±0.1, 15.3±0.1, 15.9±0.1, and 24.4±0.1° 2θ using Cu Kα radiation (e.g., 7.9, 8.7, 15.3, 15.9, and 24.4° 2θ using Cu Kα radiation))).

In some embodiments, the morphic form (e.g., Form 1) of Compound A is characterized by an XRPD pattern comprising signals (e.g., peaks) at 7.9±0.5, 8.7±0.5, 15.3±0.5, 15.9±0.5, 22.3±0.5, and 24.4±0.5° 2θ (e.g., 7.9±0.2, 8.7±0.2, 15.3±0.2, 15.9±0.2, 22.3±0.2, and 24.4±0.2° 2θ using Cu Kα radiation (e.g., 7.9±0.1, 8.7±0.1, 15.3±0.1, 15.9±0.1, 22.3±0.1, and 24.4±0.1° 2θ using Cu Kα radiation (e.g., 7.9, 8.7, 15.3, 15.9, 22.3, and 24.4° 2θ using Cu Kα radiation))).

In some embodiments, the morphic form (e.g., Form 1) of Compound A is characterized by an XRPD pattern comprising signals (e.g., peaks) at 7.9±0.5, 8.7±0.5, 15.3±0.5, 15.9±0.5, 22.3±0.5, 23.9±0.5, and 24.4±0.5° 2θ (e.g., 7.9±0.2, 8.7±0.2, 15.3±0.2, 15.9±0.2, 22.3±0.2, 23.9±0.2, and 24.4±0.2° 2θ using Cu Kα radiation (e.g., 7.9±0.1, 8.7±0.1, 15.3±0.1, 15.9±0.1, 22.3±0.1, 23.9±0.1, and 24.4±0.1° 2θ using Cu Kα radiation (e.g., 7.9, 8.7, 15.3, 15.9, 22.3, 23.9, and 24.4° 2θ using Cu Kα radiation))).

In some embodiments, the morphic form (e.g., Form 1) of Compound A is characterized by an XRPD pattern comprising signals (e.g., peaks) at 5.9±0.5, 7.9±0.5, 8.7±0.5, 15.3±0.5, 15.9±0.5, 22.3±0.5, 23.9±0.5, and 24.4±0.5° 2θ (e.g., 5.9±0.2, 7.9±0.2, 8.7±0.2, 15.3±0.2, 15.9±0.2, 22.3±0.2, 23.9±0.2, and 24.4±0.2° 2θ using Cu Kα radiation (e.g., 5.9±0.1, 7.9±0.1, 8.7±0.1, 15.3±0.1, 15.9±0.1, 22.3±0.1, 23.9±0.1, and 24.4±0.1° 2θ using Cu Kα radiation (e.g., 5.9, 7.9, 8.7, 15.3, 15.9, 22.3, 23.9, and 24.4° 2θ using Cu Kα radiation)

In some embodiments, the morphic form (e.g., Form 1) of Compound A is characterized by an XRPD pattern comprising one or more signals (e.g., peaks) as described in Table 1 below.

TABLE 1

| Angle | d Value |
|---|---|
| 5.892 * | 14.98768 Å |
| 7.943 * | 11.12115 Å |
| 8.749 * | 10.09942 Å |
| 9.600 * | 9.20520 Å |
| 12.690 * | 6.97029 Å |
| 13.739 * | 6.44006 Å |
| 14.790 * | 5.98489 Å |
| 15.348 * | 5.76828 Å |
| 15.902 * | 5.56865 Å |
| 16.498 * | 5.36874 Å |
| 17.528 * | 5.05557 Å |
| 17.757 * | 4.99097 Å |
| 18.147 * | 4.88451 Å |
| 19.271 * | 4.60210 Å |
| 19.899 * | 4.45823 Å |
| 20.390 * | 4.35200 Å |
| 20.706 * | 4.28630 Å |
| 21.787 * | 4.07604 Å |
| 22.324 * | 3.97922 Å |
| 23.920 * | 3.71707 Å |
| 24.351 * | 3.65231 Å |
| 25.050 * | 3.55203 Å |
| 25.554 * | 3.48304 Å |
| 26.178 * | 3.40145 Å |
| 26.554 * | 3.35415 Å |
| 27.362 * | 3.25690 Å |
| 27.983 * | 3.18597 Å |
| 29.418 * | 3.03377 Å |
| 29.815 * | 2.99423 Å |
| 31.060 * | 2.87697 Å |
| 32.133 * | 2.78338 Å |
| 32.750 * | 2.73234 Å |
| 33.296 * | 2.68876 Å |
| 34.847 * | 2.57252 Å |
| 35.920 * | 2.49812 Å |
| 39.478 * | 2.28077 Å |

* The values shown in the above table are approximate value and subject to instrument differentiation and standard error.

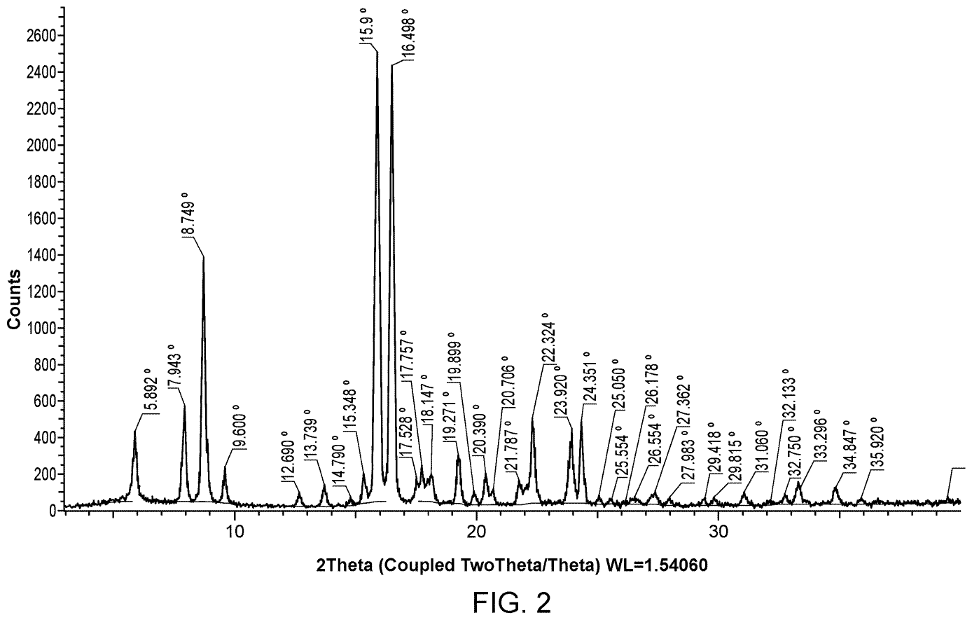

In some embodiments, the morphic form (e.g., Form 1) of Compound A is characterized by an XRPD pattern substantially similar to that set forth in FIG. 2.

### Other Characterizations

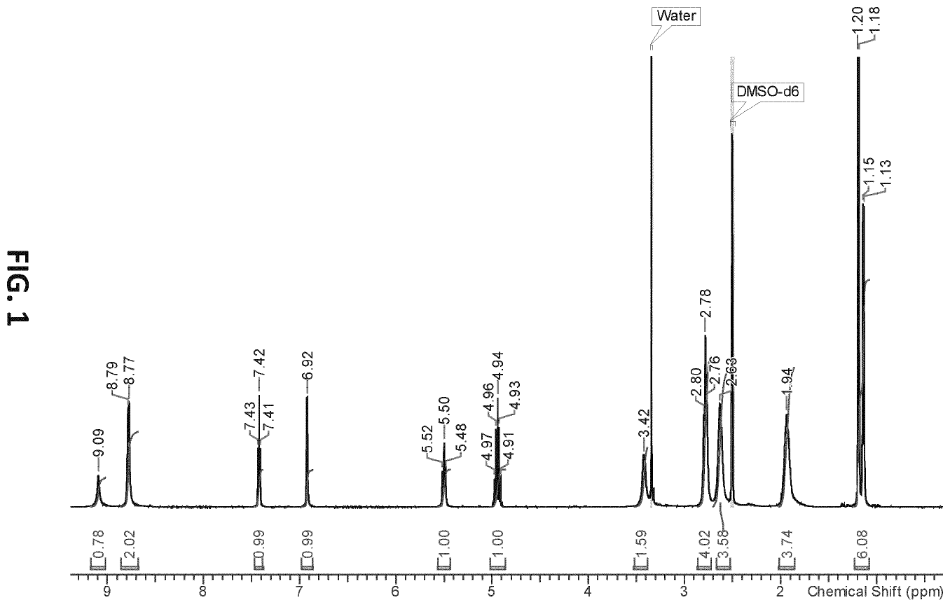

In some embodiments, the morphic form (e.g., Form 1) of Compound A is characterized by an ¹H-NMR spectrum substantially similar to that set forth in FIG. 1.

In some embodiments, the morphic form (e.g., Form 1) of Compound A is characterized by a degradation event at 220±40 °C, 220±30 °C, 220±20 °C, 220±15 °C, 220±10 °C, or 220±5 ° C (e.g., about 220 °C), as measured by thermal gravimetric analysis (TGA).

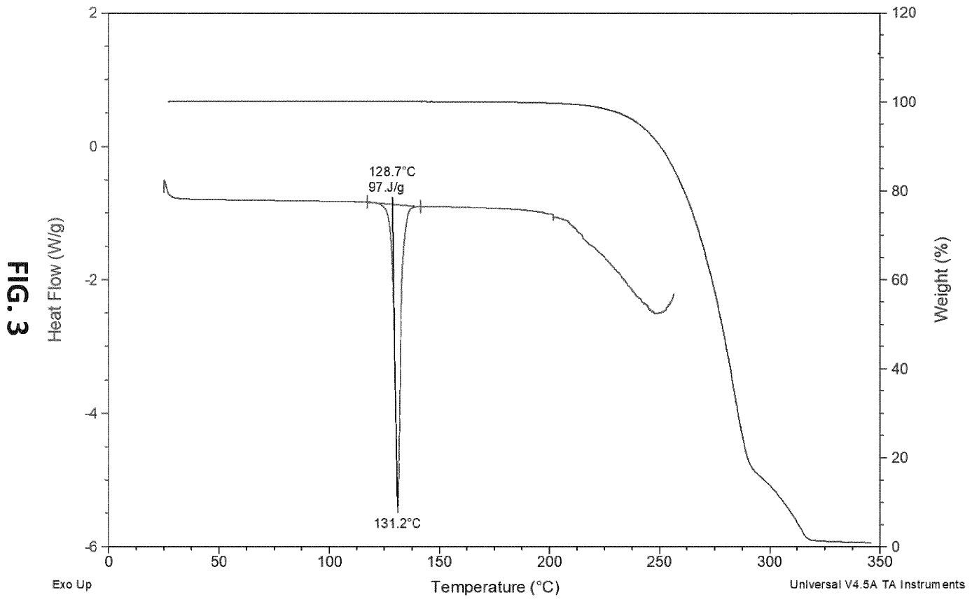

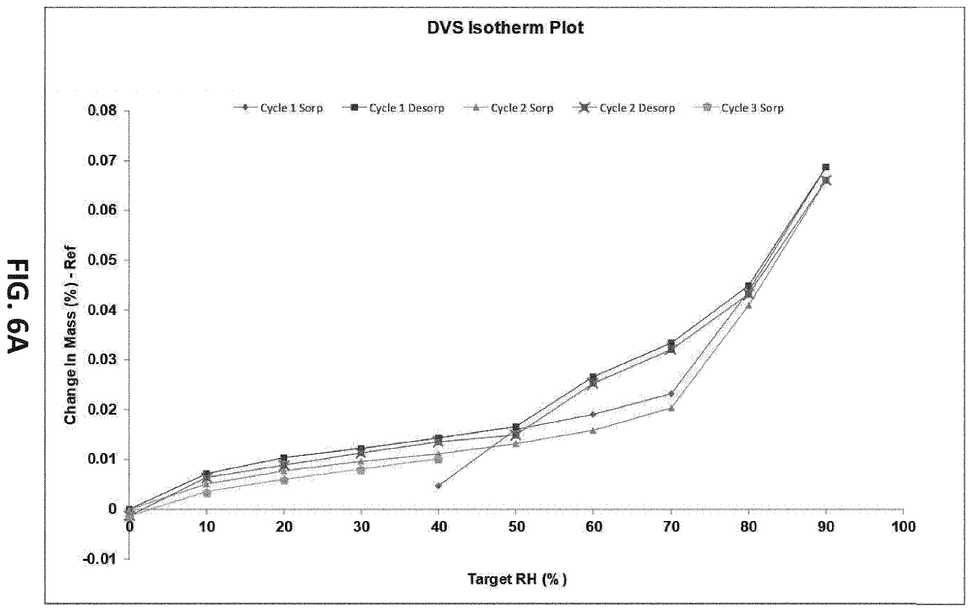

In some embodiments, the morphic form (e.g., Form 1) of Compound A is characterized by a TGA thermogram substantially similar to that set forth in FIG. 3. In some embodiments, the morphic form (e.g., Form 1) of Compound A is characterized by a Raman spectrum substantially similar to that set forth in FIG. 5.

In some embodiments, the morphic form (e.g., Form 1) of Compound A is characterized by an endothermic event with an onset at 129±20 °C, 129±15 °C, 129±10 °C, or 129±5 °C (e.g., about 129 °C), as measured by Differential Scanning calorimetry (DSC).

In some embodiments, the morphic form (e.g., Form 1) of Compound A is characterized by a water uptake (e.g., from 0% to 90% RH) of less than about 1%, less than about 0.9%, less than about 0.7%, less than about 0.6%, less than about 0.5%, less than about 0.4%, less than about 0.3%, less than about 0.2%, or less than about 0.1%, as measured by gravimetric vapour sorption (GVS).

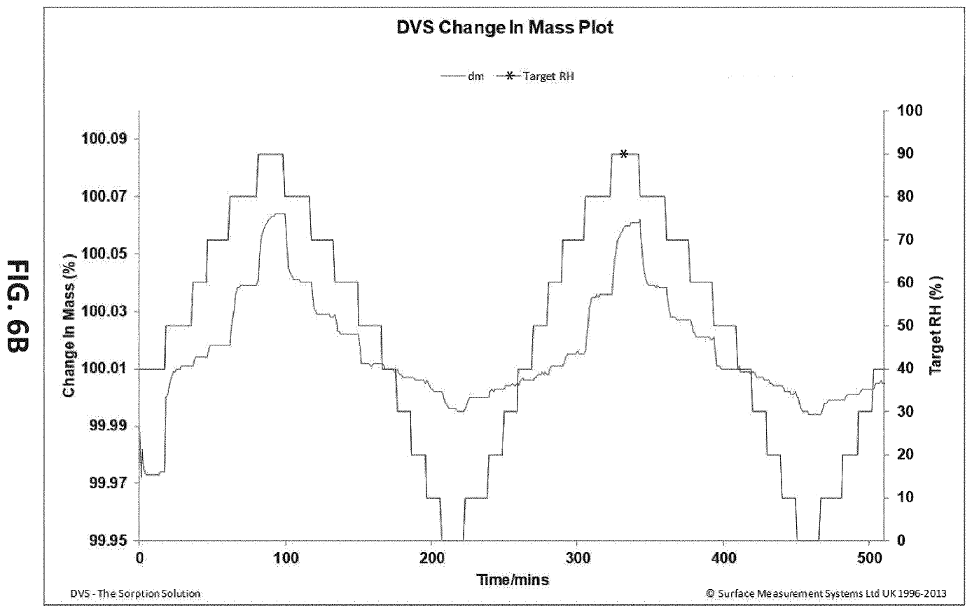

In some embodiments, the morphic form (e.g., Form 1) of Compound A is characterized by a GVS Isothermal Plot and Kinetic Plot substantially similar to that set forth in FIGS. 6A and 6B.

In some embodiments, the morphic form (e.g., Form 1) of Compound A is a crystalline solid, e.g., a needle shaped crystalline form.

In some embodiments, the morphic form (e.g., Form 1) of Compound A is a crystalline solid having an average size of 150±30 μm, 150±20 μm, 150±15 μm, 150±10 μm, or 150±5 μm (e.g., 150 μm).

### Form 2

In some embodiments, the morphic form of Compound A is Form 2 as described herein.

### X-Ray Powder Diffraction (XRPD) Characterizations

In some embodiments, the morphic form (e.g., Form 2) of Compound A is characterized by an XRPD pattern comprising signals (e.g., peaks) at 7.6±0.5, 9.4±0.5, and 15.9±0.5° 2θ (e.g., 7.6±0.2, 9.4±0.2, and 15.9±0.2° 2θ using Cu Kα radiation (e.g., 7.6±0.1, 9.4±0.1, and 15.9±0.1° 2θ using Cu Kα radiation (e.g., 7.6, 9.4, and 15.9° 2θ using Cu Kα radiation))).

In some embodiments, the morphic form (e.g., Form 2) of Compound A is characterized by an XRPD pattern comprising signals (e.g., peaks) at 7.6±0.5, 9.4±0.5, 15.2±0.5, and 15.9±0.5° 2θ (e.g., 7.6±0.2, 9.4±0.2, 15.2±0.2, and 15.9±0.2° 2θ using Cu Kα radiation (e.g., 7.6±0.1, 9.4±0.1, 15.2±0.1, and 15.9±0.1° 2θ using Cu Kα radiation (e.g., 7.6, 9.4, 15.2, and 15.9° 2θ using Cu Kα radiation))).

In some embodiments, the morphic form (e.g., Form 2) of Compound A is characterized by an XRPD pattern comprising signals (e.g., peaks) at 6.0±0.5, 7.6±0.5, 9.4±0.5, 15.2±0.5, and 15.9±0.5° 2θ (e.g., 6.0±0.2, 7.6±0.2, 9.4±0.2, 15.2±0.2, and 15.9±0.2° 2θ using Cu Kα radiation (e.g., 6.0±0.1, 7.6±0.1, 9.4±0.1, 15.2±0.1, and 15.9±0.1° 2θ using Cu Kα radiation (e.g., 6.0, 7.6, 9.4, 15.2, and 15.9° 2θ using Cu Kα radiation))).

In some embodiments, the morphic form (e.g., Form 2) of Compound A is characterized by an XRPD pattern comprising signals (e.g., peaks) at 6.0±0.5, 7.6±0.5, 9.4±0.5, 15.2±0.5, 15.9±0.5, and 21.5±0.5° 2θ (e.g., 6.0±0.2, 7.6±0.2, 9.4±0.2, 15.2±0.2, 15.9±0.2, and 21.5=0.2° 2θ using Cu Kα radiation (e.g., 6.0±0.1, 7.6±0.1, 9.4±0.1, 15.2±0.1, 15.9±0.1, and 21.5±0.1° 2θ using Cu Kα radiation (e.g., 6.0, 7.6, 9.4, 15.2, 15.9, and 21.5° 2θ using Cu Kα radiation))).

In some embodiments, the morphic form (e.g., Form 2) of Compound A is characterized by an XRPD pattern comprising signals (e.g., peaks) at 6.0±0.5, 7.6±0.5, 9.4±0.5, 15.2±0.5, 15.9±0.5, 21.5±0.5, and 23.9±0.5° 2θ (e.g., 6.0±0.2, 7.6±0.2, 9.4±0.2, 15.2±0.2, 15.9±0.2, 21.5±0.2, and 23.9±0.2° 2θ using Cu Kα radiation (e.g., 6.0±0.1, 7.6±0.1, 9.4±0.1, 15.2±0.1, 15.9±0.1, 21.5±0.1, and 23.9±0.1° 2θ using Cu Kα radiation (e.g., 6.0, 7.6, 9.4, 15.2, 15.9, 21.5, and 23.9° 2θ using Cu Kα radiation))).

In some embodiments, the morphic form (e.g., Form 2) of Compound A is characterized by an XRPD pattern comprising signals (e.g., peaks) at 6.0±0.5, 7.6±0.5, 9.4±0.5, 15.2±0.5, 15.9±0.5, 21.2±0.5, 21.5±0.5, and 23.9±0.5° 2θ (e.g., 6.0±0.2, 7.6±0.2, 9.4±0.2, 15.2±0.2, 15.9±0.2, 21.2±0.2, 21.5±0.2, and 23.9±0.2° 2θ using Cu Kα radiation (e.g., 6.0±0.1, 7.6±0.1, 9.4±0.1, 15.2±0.1, 15.9±0.1, 21.2±0.1, 21.5±0.1, and 23.9±0.1°20 using Cu Kα radiation (e.g., 6.0, 7.6, 9.4, 15.2, 15.9, 21.2, 21.5, and 23.9° 2θ using Cu Kα radiation))).

In some embodiments, the morphic form (e.g., Form 2) of Compound A is characterized by an XRPD pattern comprising one or more signals (e.g., peaks) as described in Table 2 below.

TABLE 2

| Angle | d Value |
|---|---|
| 6.014 * | 14.68445 Å |
| 7.565 * | 11.67742 Å |
| 8.743 * | 10.10588 Å |
| 9.369 * | 9.43181 Å |
| 10.110 * | 8.74191 Å |
| 14.631 * | 6.04957 Å |
| 15.157 * | 5.84074 Å |
| 15.879 * | 5.57676 Å |
| 16.464 * | 5.37988 Å |
| 17.849 * | 4.96530 Å |
| 18.145 * | 4.88503 Å |
| 18.802 * | 4.71586 Å |
| 19.200 * | 4.61889 Å |
| 19.562 * | 4.53431 Å |
| 19.919 * | 4.45380 Å |
| 20.290 * | 4.37330 Å |
| 20.721 * | 4.28318 Å |
| 21.208 * | 4.18591 Å |
| 21.538 * | 4.12252 Å |
| 22.343 * | 3.97582 Å |
| 22.829 * | 3.89222 Å |
| 23.191 * | 3.83229 Å |
| 23.876 * | 3.72396 Å |
| 24.742 * | 3.59544 Å |
| 26.252 * | 3.39198 Å |
| 27.108 * | 3.28681 Å |
| 28.343 * | 3.14637 Å |
| 29.506 * | 3.02487 Å |
| 29.769 * | 2.99873 Å |
| 30.291 * | 2.94824 Å |
| 32.390 * | 2.76182 Å |
| 33.798 * | 2.64995 Å |
| 35.895 * | 2.49979 Å |
| 37.591 * | 2.39081 Å |
| 38.537 * | 2.33427 Å |

* The values shown in the above table are approximate value and subject to instrument differentiation and standard error.

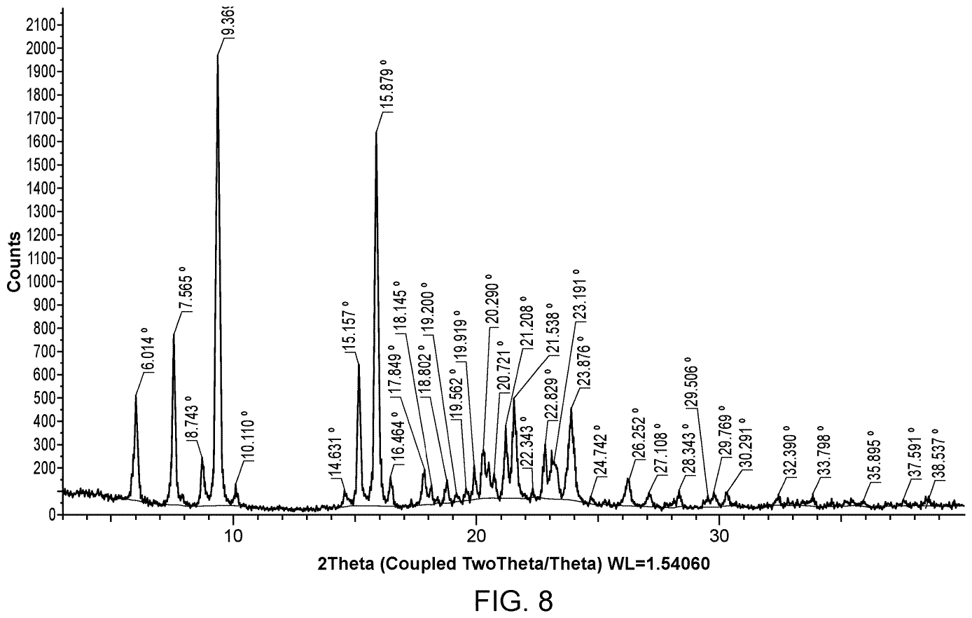

In some embodiments, the morphic form (e.g., Form 2) of Compound A is characterized by an XRPD pattern substantially similar to that set forth in FIG. 8.

### Other Characterizations

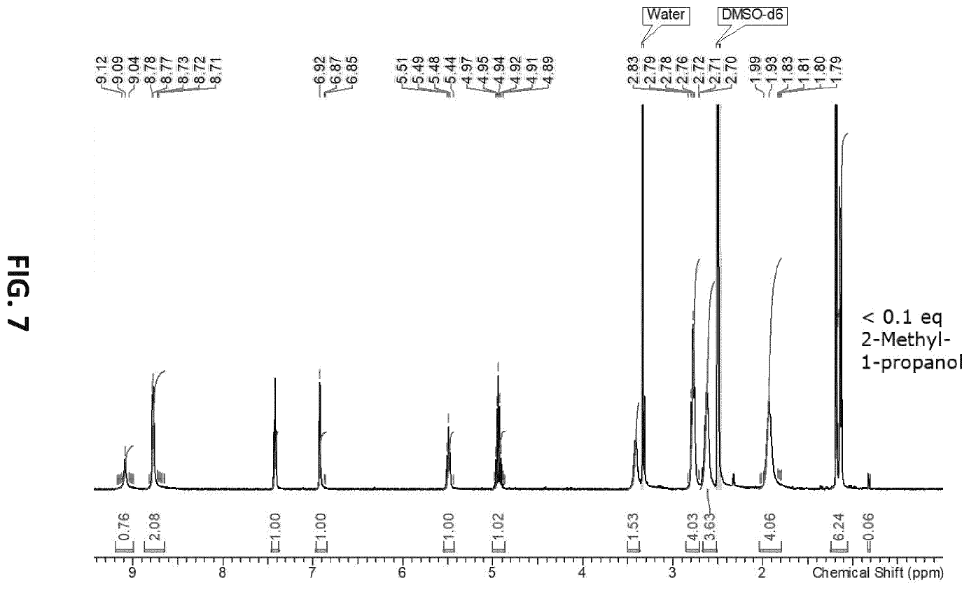

In some embodiments, the morphic form (e.g., Form 2) of Compound A is characterized by a ¹H-NMR spectrum substantially similar to that set forth in FIG. 7.

In some embodiments, the morphic form (e.g., Form 2) of Compound A is characterized by a degradation event at 200±40 °C, 200±30 °C, 200±20 °C, 200±15 °C, 200±10 °C, or 200-5 °C (e.g., about 200 °C), as measured by thermal gravimetric analysis (TGA).

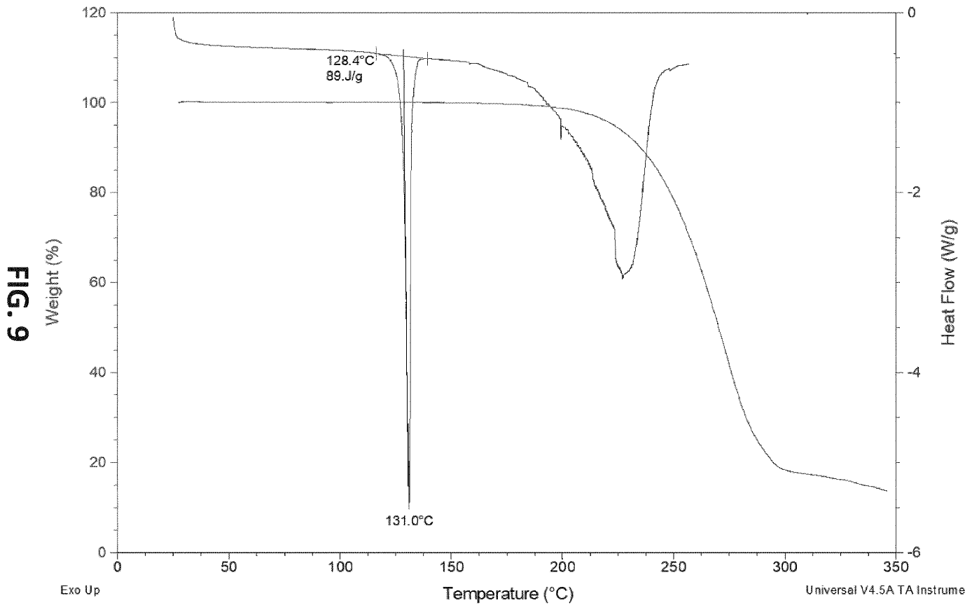

In some embodiments, the morphic form (e.g., Form 2) of Compound A is characterized by a TGA thermogram substantially similar to that set forth in FIG. 9.

In some embodiments, the morphic form (e.g., Form 2) of Compound A is characterized by an endothermic event with an onset at 129±20 °C, 129±15 °C, 129±10 °C, or 129±5 °C (e.g., about 129 °C), as measured by Differential Scanning calorimetry (DSC).

In some embodiments, the morphic form (e.g., Form 2) of Compound A is characterized by a water uptake (e.g., from 0% to 90% RH) of 0.15±0.5%, 0.15±0.4%, 0.15±0.3%, 0.15±0.2%, 0.15±0.1%, or 0.15±0.05% (e.g., about 0.15%), as measured by gravimetric vapour sorption (GVS).

In some embodiments, the morphic form (e.g., Form 2) of Compound A is characterized by a water uptake (e.g., from 40% to 90% RH) of 0.09±0.05%, 0.09±0.04%, 0.09±0.03%, 0.09±0.02%, or 0.09±0.01% (e.g., about 0.09%), as measured by gravimetric vapour sorption (GVS).

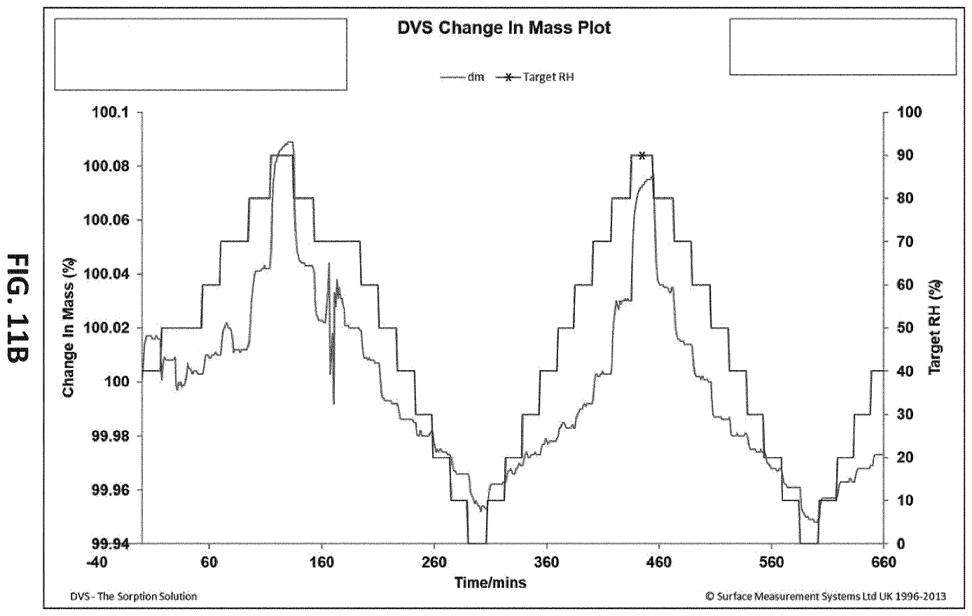

In some embodiments, the morphic form (e.g., Form 2) of Compound A is characterized by a GVS Isothermal Plot and Kinetic Plot substantially similar to that set forth in FIGS. 11A and 11B.

In some embodiments, the morphic form (e.g., Form 2) of Compound A is a crystalline solid, e.g., a needle shaped crystalline form.

In some embodiments, the morphic form (e.g., Form 2) of Compound A is a crystalline solid having an average size ranging from about 50 μm to about 75 μm.

In some embodiments, the morphic form (e.g., Form 2) of Compound A is a crystalline solid having an average size of 68±30 μm, 68±20 μm, 68±15 μm, 68±10 μm, or 68±5 μm (e.g., 68 μm).

### Other Properties of the Crystalline Forms

In some embodiments, the morphic form of Compound A has a purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

In some embodiments, Form 1 of Compound A has a purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater as measured by HPLC.

In some embodiments, Form 2 of Compound A has a purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater as measured by HPLC.

In some embodiments, the morphic form of Compound A has a morphic purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

In some embodiments, Form 1 of Compound A has a morphic purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater as measured by HPLC.

In some embodiments, Form 2 of Compound A has a morphic purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater as measured by HPLC.

In some embodiments, the morphic form of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 25 °C and 97% relative humidity (RH), over 1 day, 2 days, 3 days, 4 days, 5 days, 6 days, one week, two weeks, three weeks, one month, two months, three months, or four months.

In some embodiments, Form 1 of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity as measured by HPLC, under 25 °C and 97% relative humidity (RH), over 1 day, 2 days, 3 days, 4 days, 5 days, 6 days, one week, two weeks, three weeks, one month, two months, three months, or four months.

In some embodiments, Form 2 of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity as measured by HPLC, under 25 °C and 97% relative humidity (RH), over 1 day, 2 days, 3 days, 4 days, 5 days, 6 days, one week, two weeks, three weeks, one month, two months, three months, or four months.

In some embodiments, the morphic form of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 40 °C and 75% relative humidity (RH), over 1 day, 2 days, 3 days, 4 days, 5 days, 6 days, one week, two weeks, three weeks, one month, two months, three months, or four months.

In some embodiments, Form 1 of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity as measured by HPLC, under 40 °C and 75% relative humidity (RH), over 1 day, 2 days, 3 days, 4 days, 5 days, 6 days, one week, two weeks, three weeks, one month, two months, three months, or four months.

In some embodiments, Form 2 of Compound A exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity as measured by HPLC, under 40 °C and 75% relative humidity (RH), over 1 day, 2 days, 3 days, 4 days, 5 days, 6 days, one week, two weeks, three weeks, one month, two months, three months, or four months.

### Methods of Preparing the Crystalline Forms

In some aspects, the present disclosure provides a method of preparing a crystalline form of Compound A described herein.

In some aspects, the present disclosure provides a method of preparing a crystalline form of Compound A, comprising one or more steps as described herein.

In some aspects, the present disclosure provides a compound obtainable by, or obtained by, or directly obtained by a method for preparing a crystalline form of Compound A as described herein.

In some embodiments, the prepared crystalline form of Compound A has a higher purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater).

In some embodiments, the prepared crystalline Form 1 of Compound A has a higher purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater as measured by HPLC).

In some embodiments, the prepared crystalline Form 2 of Compound A has a higher purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater as measured by HPLC).

In some embodiments, the prepared crystalline form of Compound A (e.g., Form 1 or Form 2) has a higher morphic purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater).

In some embodiments, the prepared crystalline Form 1 of Compound A has a higher morphic purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater as measured by HPLC).

In some embodiments, the prepared crystalline Form 2 of Compound A has a higher morphic purity as compared to Compound A being prepared by a comparable method (e.g., by 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, or 50% or greater as measured by HPLC).

In some embodiments, the morphic form of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50%), under 25 °C and 97% relative humidity (RH), over 1 day, 2 days, 3 days, 4 days, 5 days, 6 days, one week, two weeks, three weeks, one month, two months, three months, or four months.

In some embodiments, Form 1 of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50% as measured by HPLC), under 25 °C and 97% relative humidity (RH), over 1 day, 2 days, 3 days, 4 days, 5 days, 6 days, one week, two weeks, three weeks, one month, two months, three months, or four months.

In some embodiments, Form 2 of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50% as measured by HPLC), under 25 °C and 97% relative humidity (RH), over 1 day, 2 days, 3 days, 4 days, 5 days, 6 days, one week, two weeks, three weeks, one month, two months, three months, or four months.

In some embodiments, the morphic form of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50%), under 40 °C and 75% relative humidity (RH), over 1 day, 2 days, 3 days, 4 days, 5 days, 6 days, one week, two weeks, three weeks, one month, two months, three months, or four months.

In some embodiments, Form 1 of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50% as measured by HPLC), under 40 °C and 75% relative humidity (RH), over 1 day, 2 days, 3 days, 4 days, 5 days, 6 days, one week, two weeks, three weeks, one month, two months, three months, or four months.

In some embodiments, Form 2 of Compound A exhibits less impurity as compared to Compound A being prepared by a comparable method (e.g., by about 1%, about 2%, about 3%, about 4%, about 5%, about 10%, about 15%, about 20%, about 25%, about 30%, about 35%, about 40%, about 45%, or about 50% as measured by HPLC), under 40 °C and 75% relative humidity (RH), over 1 day, 2 days, 3 days, 4 days, 5 days, 6 days, one week, two weeks, three weeks, one month, two months, three months, or four months.

The crystalline form of Compound A can be prepared by any suitable technique known in the art. Particular processes for the preparation of these compounds are described further in the accompanying examples.

In some embodiments, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between about 5 minutes and about 48 hours.

### Biological Assays

Compounds described herein can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological away is a biological away testing inhibitory activity against IL-1β release upon NLRP3 activation in peripheral blood mononuclear cells (PBMC).

In some embodiments, the biological assay is a PBMC IC50 Determination Assay.

In some embodiments, the compound is tested for their inhibitory activity against IL-1β release upon NLRP3 activation in blood cells (e.g., peripheral blood mononuclear cells (PBMC)).

In some embodiments, PBMC is isolated and seeded into the wells of a plate and incubated for a period of time (e.g., for 3 hours with a lipopolysaccharide). Following incubation, the medium is exchanged and the compound added to the well (e.g., a compound of the present disclosure) and the cells may be incubated. Next, the cells are stimulated (e.g., with ATP or nigericin) and the cell culture media are collected for analysis.

In some embodiments, the release of IL-1β into the media is determined by a quantitative detection of IL-1β in the media (e.g., using ELISA).

In some embodiments, PBMC is isolated (e.g., from buffy coats). Isolated cells are seeded into wells and incubated (e.g., for 3 hours with lipopolysaccharide). The compound is then be added and the cells incubated. Next, the cells are stimulated and the media from the wells are collected for analysis.

In some embodiments, the release of IL-1β into the media is determined by quantitative detection (e.g., of IL-1β in media using HTRF®).

### Pharmaceutical Compositions

In some aspects, the present disclosure provides pharmaceutical compositions comprising a crystalline form of Compound A described herein, and one or more pharmaceutically acceptable carrier or excipient.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carrier comprising one or more excipient and/or auxiliary that facilitates processing of the active compound into preparations that can be used pharmaceutically. A person of skill in the art would understand that the appropriate formulation may be dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers may include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions may include one or more inert diluent or one or more edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with one or more pharmaceutically acceptable carrier that may protect the compound against rapid elimination from the body, such as a controlled release formulation, including an implant and microencapsulated delivery system. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease.

It is understood that the pharmaceutical compositions can be included in a container, pack, or dispenser together with instructions for administration.

### Methods of Use

In some aspects, the present disclosure provides a method of preventing or treating a disease or disorder in a subject, comprising administering to the subject a pharmaceutically effective amount of a morphic form of Compound A described herein.

In some aspects, the present disclosure provides a method of treating a disease or disorder in a subject, comprising administering to the subject a pharmaceutically effective amount of a morphic form of Compound A described herein.

In some aspects, the present disclosure provides a morphic form of Compound A described herein for use in preventing or treating a disease or disorder in a subject.

In some aspects, the present disclosure provides a morphic form of Compound A described herein for use in treating a disease or disorder in a subject.

In some aspects, the present disclosure provides use of a morphic form of Compound A described herein in the manufacture of a medicament for preventing or treating a disease or disorder in a subject.

In some aspects, the present disclosure provides use of a morphic form of Compound A described herein in the manufacture of a medicament for treating a disease or disorder in a subject.

In some aspects, the present disclosure provides use of a morphic form of Compound A described herein for preventing or treating a disease or disorder in a subject.

In some aspects, the present disclosure provides use of a morphic form of Compound A described herein for treating a disease or disorder in a subject.

In some embodiments, the disease or disorder is associated with an implicated inflammasome activity. In some embodiments, the disease or disorder is a disease or disorder in which inflammasome activity is implicated.

In some embodiments, the disease or disorder is an inflammatory disorder, autoinflammatory disorder, an autoimmune disorder, a neurodegenerative disease, or cancer.

In some embodiments, the disease or disorder is an inflammatory disorder, autoinflammatory disorder and/or an autoimmune disorder.

In some embodiments, the disease or disorder is selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases).

In some embodiments, the disease or disorder is a neurodegenerative disease.

In some embodiments, the disease or disorder is Parkinson's disease or Alzheimer's disease.

In some embodiments, the disease or disorder is a dermatological disease.

In some embodiments, the dermatological disease is acne.

In some embodiments, the disease or disorder is cancer.

In some embodiments, the cancer is metastasising cancer, gastrointestinal cancer, skin cancer, non-small-cell lung carcinoma, brain cancer (e.g. glioblastoma) or colorectal adenocarcinoma.

In some aspects, the present disclosure provides a method of inhibiting inflammasome (e.g., the NLRP3 inflammasome) activity in a subject (e.g., in vitro or in vivo), comprising contacting a cell with an effective amount of a crystalline form of Compound A.

In some aspects, the present disclosure provides a crystalline form of Compound A described herein for use in inhibiting inflammasome (e.g., the NLRP3 inflammasome) activity in a subject (e.g., in vitro or in vivo).

In some aspects, the present disclosure provides use of a crystalline form of Compound A described herein in the manufacture of a medicament for inhibiting inflammasome (e.g., the NLRP3 inflammasome) activity (e.g., in vitro or in vivo).

In some embodiments, the inflammasome is NLRP3 inflammasome.

In some embodiments, the subject is an animal.

In some embodiments, the subject is a mammal.

In some embodiments, the subject is a human.

In some embodiments, the subject is a cell.

In some embodiments, the subject is a cell population.

### DEFINITIONS

Unless otherwise stated, the following terms used in the specification and claims have the following meanings set out below.

As used herein, the term “about” means approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 20%, a variance of 10%, a variance of 5%, a variance of 3%, or a variance of 1%.

It is understood that the compounds of the present disclosure can exist in either hydrated or unhydrated (the anhydrous) form or as solvates with other solvent molecules. Nonlimiting examples of hydrates may include monohydrates and dihydrates. Nonlimiting examples of solvates may include ethanol solvates and acetone solvates.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of a crystalline form of Compound A.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used.

It is to be understood that a crystalline form of Compound A can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic scheme, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of a crystalline form of Compound A to provide such treatment or prevention as is described herein. It is to be further understood, unless otherwise stated, any description of a method of treatment or prevention includes use of a crystalline form of Compound A to prepare a medicament to treat or prevent such condition. The treatment or prevention includes treatment or prevention of human or non-human animals including rodents and other disease models.

It is to be understood that, unless otherwise stated, any description of a method of treatment includes use of a crystalline form of Compound A to provide such treatment as is described herein. It is to be further understood, unless otherwise stated, any description of a method of treatment includes use of a crystalline form of Compound A to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a morphic form of Compound A, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that “solubility” or “solubility rating” refers to the property of a polymorph disclosed herein to dissolve in a liquid solvent and form a homogeneous solution. In some embodiments, solubility is expressed as a concentration, either by mass of solute per unit volume of solvent (e.g., g of solute per kg of solvent, g per dL (100 mL), mg/ml, etc.), molarity, molality, mole fraction, or other similar descriptions of concentration. A person of skill in the art may understand that the maximum equilibrium amount of solute that can dissolve per amount of solvent is the solubility of that solute in that solvent under the specified conditions, including temperature, pressure, pH, and the nature of the solvent. In some embodiments, solubility is measured at physiological pH, or non-physiological pH, for example, at about pH 5.0, about pH 6.0, about pH 7.0, about pH 7.4, about pH 7.6, about pH 7.8, or about pH 8.0 (e.g., about pH 5-8). In some embodiments, solubility is measured in water or a physiological buffer, for example PBS, NaCl (with or without NaPO₄), or FaSSIF. In some embodiments, solubility is measured in a biological fluid (solvent) (e.g., blood or serum). In some embodiments, the temperature is be about room temperature (e.g., about 20, about 21, about 22, about 23, about 24, or about 25 °C) or about body temperature (about 37 °C). In some embodiments, an agent has a solubility rating of at least about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 40, about 50, about 60, about 70, about 80, about 90 or about 100 mg/ml at room temperature or at 37 °C.

As used herein, “stable” refers to a polymorph that maintains purity, appearance, and/or analytical parameters over a defined time and temperature as compared to the polymorph as isolated. In some embodiments, the “stable” polymorph exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity over a set period of time (e.g., 1 day, 2 days, 3 days, 4 days, 5 days, 6 days, one week, two weeks, three weeks, one month, two months, three months, or four months). For example, a polymorph is stable if after two weeks at room temperature the DSC and TGA profiles are consistent with the originally isolated polymorph.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising a crystalline form of Compound A in combination with at least one pharmaceutically acceptable excipient or carrier.

As used herein, the term “pharmaceutical composition” is a formulation containing a crystalline form of Compound A in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with one or more pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims may include both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount or effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing a crystalline form of Compound A may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carrier comprising excipients and/or auxiliaries that facilitate processing of a crystalline form of Compound A into preparations that can be used pharmaceutically. The appropriate formulation is dependent upon the route of administration chosen.

The crystalline form of Compound A can be prepared with one or more pharmaceutically acceptable carrier that will protect the compound against rapid elimination from the body, such as a controlled release formulation, including an implant and microencapsulated delivery system.

It is to be understood that the pharmaceutical compositions can be included in a container, pack, or dispenser together with instructions for administration.

Techniques for formulation and administration of the disclosed compounds of the disclosure can be found in Remington: the Science and Practice of Pharmacy, 19^{th} edition, Mack Publishing Co., Easton, PA (1995). In some embodiments, a crystalline form of Compound A, is used in pharmaceutical preparations in combination with one or more pharmaceutically acceptable carrier or diluent. A suitable pharmaceutically acceptable carrier includes, but is not limited to, inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic scheme, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

The disclosure having been described, the following examples are offered by way of illustration and not limitation.

### EXEMPLARY EMBODIMENTS

Exemplary Embodiment 1. A morphic form of Compound A:

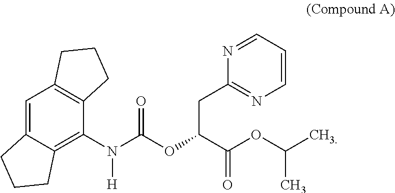 [SMILES: CC(C)OC(=O)[C@@H](Cc1ncccn1)OC(=O)Nc1c2c(cc3c1CCC3)CCC2]

Exemplary Embodiment 2. The morphic form of Exemplary Embodiment 1, being a crystalline form of Compound A.

Exemplary Embodiment 3. The morphic form of any one of the preceding Exemplary Embodiments, being Form 1 of Compound A.

Exemplary Embodiment 4. The morphic form of any one of the preceding Exemplary Embodiments, wherein the morphic form is characterized by an XRPD pattern comprising signals at 8.7±0.5, 15.3±0.5, and 15.9±0.5° 2θ using Cu Kα radiation.

Exemplary Embodiment 5. The morphic form of any one of the preceding Exemplary Embodiments, wherein the morphic form is characterized by an XRPD pattern comprising signals at 7.9±0.5, 8.7±0.5, 15.3±0.5, and 15.9±0.5° 2θ using Cu Kα radiation.

Exemplary Embodiment 6. The morphic form of any one of the preceding Exemplary Embodiments, wherein the morphic form is characterized by an XRPD pattern comprising signals at 7.9±0.5, 8.7±0.5, 15.3±0.5, 15.9±0.5, and 24.4±0.5° 2θ using Cu Kα radiation.

Exemplary Embodiment 7. The morphic form of any one of the preceding Exemplary Embodiments, wherein the morphic form is characterized by an XRPD pattern comprising signals at 7.9±0.5, 8.7±0.5, 15.3±0.5, 15.9±0.5, 22.3±0.5, and 24.4±0.5° 2θ using Cu Kα radiation.

Exemplary Embodiment 8. The morphic form of any one of the preceding Exemplary Embodiments, wherein the morphic form is characterized by an XRPD pattern comprising signals at 7.9±0.5, 8.7±0.5, 15.3±0.5, 15.9±0.5, 22.3±0.5, 23.9±0.5, and 24.4±0.5° 2θ using Cu Kα radiation.

Exemplary Embodiment 9. The morphic form of any one of the preceding Exemplary Embodiments, wherein the morphic form is characterized by an XRPD pattern comprising signals at 5.9±0.5, 7.9±0.5, 8.7±0.5, 15.3±0.5, 15.9±0.5, 22.3±0.5, 23.9±0.5, and 24.4±0.5° 2θ using Cu Kα radiation.

Exemplary Embodiment 10. The morphic form of any one of the preceding Exemplary Embodiments, wherein the morphic form is characterized by an XRPD pattern comprising one or more signals as described in Table 1.

Exemplary Embodiment 11. The morphic form of any one of the preceding Exemplary Embodiments, wherein the morphic form is characterized by an XRPD pattern substantially similar to that set forth in FIG. 2.

Exemplary Embodiment 12. The morphic form of any one of Exemplary Embodiments 1-2, being Form 2 of Compound A.

Exemplary Embodiment 13. The morphic form of any one of Exemplary Embodiments 1-2 and 12, wherein the morphic form is characterized by an XRPD pattern comprising signals at 7.6±0.5, 9.4±0.5, and 15.9±0.5° 2θ using Cu Kα radiation.

Exemplary Embodiment 14. The morphic form of any one of Exemplary Embodiments 1-2 and 12-13, wherein the morphic form is characterized by an XRPD pattern comprising signals at 7.6±0.5, 9.4±0.5, 15.2±0.5, and 15.9±0.5° 2θ using Cu Kα radiation.

Exemplary Embodiment 15. The morphic form of any one of Exemplary Embodiments 1-2 and 12-14, wherein the morphic form is characterized by an XRPD pattern comprising signals at 6.0±0.5, 7.6±0.5, 9.4±0.5, 15.2±0.5, and 15.9±0.5° 2θ using Cu Kα radiation.

Exemplary Embodiment 16. The morphic form of any one of Exemplary Embodiments 1-2 and 12-15, wherein the morphic form is characterized by an XRPD pattern comprising signals at 6.0±0.5, 7.6±0.5, 9.4±0.5, 15.2±0.5, 15.9±0.5, and 21.5±0.5° 2θ using Cu Kα radiation.

Exemplary Embodiment 17. The morphic form of any one of Exemplary Embodiments 1-2 and 12-16, wherein the morphic form is characterized by an XRPD pattern comprising signals at 6.0±0.5, 7.6±0.5, 9.4±0.5, 15.2±0.5, 15.9±0.5, 21.5±0.5, and 23.9±0.5° 2θ using Cu Kα radiation.

Exemplary Embodiment 18. The morphic form of any one of Exemplary Embodiments 1-2 and 12-17, wherein the morphic form is characterized by an XRPD pattern comprising signals at 6.0±0.5, 7.6±0.5, 9.4±0.5, 15.2±0.5, 15.9±0.5, 21.2±0.5, 21.5±0.5, and 23.9±0.5° 2θ using Cu Kα radiation.

Exemplary Embodiment 19. The morphic form of any one of Exemplary Embodiments 1-2 and 12-18, wherein the morphic form is characterized by an XRPD pattern comprising one or more signals as described in Table 2.

Exemplary Embodiment 20. The morphic form of any one of Exemplary Embodiments 1-2 and 12-19, wherein the morphic form is characterized by an XRPD pattern substantially similar to that set forth in FIG. 8.

Exemplary Embodiment 21. The morphic form of any one of the preceding Exemplary Embodiments, having a purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

Exemplary Embodiment 22. The morphic form of any one of the preceding Exemplary Embodiments, having a morphic purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

Exemplary Embodiment 23. The morphic form of any one of the preceding Exemplary Embodiments, exhibiting less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 25 °C and 97% relative humidity (RH), over 1 day, 2 days, 3 days, 4 days, 5 days, 6 days, one week, two weeks, three weeks, one month, two months, three months, or four months.

Exemplary Embodiment 24. The morphic form of any one of the preceding Exemplary Embodiments, exhibiting less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 40 °C and 75% relative humidity (RH), over 1 day, 2 days, 3 days, 4 days, 5 days, 6 days, one week, two weeks, three weeks, one month, two months, three months, or four months.

Exemplary Embodiment 25. A method of preparing a morphic form of Compound A of any one of the preceding Exemplary Embodiments.

Exemplary Embodiment 26. A pharmaceutical composition comprising a morphic form of Compound A of any one of the preceding Exemplary Embodiments, and one or more pharmaceutically acceptable carrier or excipient.

Exemplary Embodiment 27. A method of preventing or treating a disease or disorder in a subject, comprising administering to the subject the morphic form of Compound A of any one of Exemplary Embodiments 1-24 or the pharmaceutical composition of Exemplary Embodiment 26.

Exemplary Embodiment 28. The morphic form of Compound A of any one of Exemplary Embodiments 1-24 or the pharmaceutical composition of Exemplary Embodiment 26 for use in preventing or treating a disease or disorder in a subject.

Exemplary Embodiment 29. Use of the morphic form of Compound A of any one of Exemplary Embodiments 1-24 or the pharmaceutical composition of Exemplary Embodiment 26 in the manufacture of a medicament for preventing or treating a disease or disorder in a subject.

Exemplary Embodiment 30. A method of inhibiting inflammasome activity in a subject, comprising contacting a cell with the morphic form of Compound A of any one of Exemplary Embodiments 1-24 or the pharmaceutical composition of Exemplary Embodiment 26.

Exemplary Embodiment 31. The morphic form of Compound A of any one of Exemplary Embodiments 1-24 or the pharmaceutical composition of Exemplary Embodiment 26 for use in inhibiting inflammasome activity in a subject.

Exemplary Embodiment 32. Use of a morphic form of Compound A in the manufacture of a medicament for inhibiting inflammasome activity in a subject.

Exemplary Embodiment 33. The method, morphic form, pharmaceutical composition, or use of any one of the preceding Exemplary Embodiments, wherein the subject is human.

### EXAMPLES

### Instrument and Methodology Details

The instrument and methodology details for the experiments carried out in the examples are described herein.

### X-Ray Powder Diffraction (XRPD)

### Bruker AXS C2 GADDS:

XRPD diffractograms were collected on a Bruker AXS C2 GADDS diffractometer using Cu Kα radiation (40 kV, 40 mA), an automated XYZ stage, a laser video microscope for auto-sample positioning and a Våntec-500 2-dimensional area detector. X-ray optics consists of a single Göbel multilayer mirror coupled with a pinhole collimator of 0.3 mm. The beam divergence, i.e. the effective size of the X-ray beam on the sample, was approximately 4 mm. A θ-θ continuous scan mode was employed with a sample-detector distance of 20 cm which gives an effective 20 range of 1.5°-32.5°. Typically, the sample was exposed to the X-ray beam for 120 seconds. The software used for data collection and analysis was GADDS for Win7/XP and Diffrac Plus EVA respectively.

Ambient conditions: Samples run under ambient conditions were prepared as flat plate specimens using powder as received without grinding. Samples were prepared and analysed on a glass slide, by lightly pressed the powder to obtain a flat surface for analysis.

Non-ambient conditions: For variable temperature (VT-XRPD) experiments samples were mounted on an Anton Paar DHS 900 hot stage at ambient conditions. The sample was then heated to the appropriate temperature at 20 °C/min and subsequently held isothermally for 1 minute before data collection. Samples were prepared and analysed on a silicon wafer mounted to the hot stage using a heat-conducting paste.

### Bruker AXS D8 Advance:

XRPD diffractograms were collected on a Bruker D8 diffractometer using Cu Kα radiation (40 kV, 40 mA) and a 0-20 goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard Pharmorphix data collection method are:
Angular range: 2 to 42° 2θ
Step size: 0.05° 2θ
Collection time: 0.5 s/step (total collection time: 6.40 min)

When required other methods for data collection are used with details as follows:

4 Minute Method

Angular range: 2 to 31° 2θ
Step size: 0.06° 2θ
Time per step: 0.5 s/step

### PANalytical Empyrean:

XRPD diffractograms were collected on a PANalytical Empyrean diffractometer using Cu Kα radiation (45 kV, 40 mA) in transmission geometry. A 0.5° slit, 4 mm mask and 0.04 rad Soller slits with a focusing mirror were used on the incident beam. A PIXcel^{3D} detector, placed on the diffracted beam, was fitted with a receiving slit and 0.04 rad Soller slits. The software used for data collection was X′Pert Data Collector using X′Pert Operator Interface. The data were analysed and presented using Diffrac Plus EVA or HighScore Plus. Samples were prepared and analysed in either a metal or Millipore 96 well-plate in transmission mode. X-ray transparent film was used between the metal sheets on the metal well-plate and powders (approximately 1-2 mg) were used as received. The Millipore plate was used to isolate and analyse solids from suspensions by adding a small amount of suspension directly to the plate before filtration under a light vacuum.

The scan mode for the metal plate used the gonio scan axis, whereas a 20 scan was utilised for the Millipore plate.

The details of the standard screening data collection method are:
Angular range: 2.5 to 32.0° 2θ
Step size: 0.0130° 2θ
Collection time: 12.75 s/step (total collection time of 2.07 min)

### Nuclear Magnetic Resonance (NMR)

¹H NMR spectra were collected on a Bruker 400 MHz instrument equipped with an auto-sampler and controlled by a DRX400 console. Samples were prepared in DMSO-d₆ solvent, unless otherwise stated. Automated experiments were acquired using ICON-NMR configuration within Topspin software, using standard Bruker-loaded experiments (¹H). Off-line analysis was performed using ACD Spectrus Processor.

### Differential Scanning Calorimetry (DSC)

TA Instruments Q2000: DSC data were collected on a TA Instruments Q2000 equipped with a 50 position auto-sampler. Typically, 0.5-3 mg of each sample, in a pin-holed aluminium pan, was heated at 10 °C/min from 25 °C to 260 °C. A purge of dry nitrogen at 50 ml/min was maintained over the sample. Modulated temperature DSC was carried out using an underlying heating rate of 2 °C/min and temperature modulation parameters of ±0.636 °C (amplitude) every 60 seconds (period). The instrument control software was Advantage for Q Series and Thermal Advantage and the data were analysed using Universal Analysis or TRIOS.

### Thermo-Gravimetric Analysis (TGA)

TA Instruments Q500: TGA data were collected on a TA Instruments Q500 TGA, equipped with a 16 position auto-sampler. Typically, 5-10 mg of each sample was loaded onto a pre-tared aluminium DSC pan and heated at 10 °C/min from ambient temperature to 350 °C. A nitrogen purge at 60 ml/min was maintained over the sample. The instrument control software was Advantage for Q Series and Thermal Advantage and the data were analysed using Universal Analysis or TRIOS.

### Polarised Light Microscopy (PLM)

Leica LM/DM Polarised Light Microscope: Samples were analysed on a Leica LM/DM polarised light microscope with a digital video camera for image capture. A small amount of each sample was placed on a glass slide, with or without immersion oil, and covered with a glass slip. The sample was viewed with appropriate magnification and partially polarised light, coupled to a λ false-colour filter. Images were captured using StudioCapture or Image ProPlus software.

### Scanning Electron Microscopy (SEM)

Data were collected on a Phenom Pro Scanning Electron Microscope. A small quantity of sample was mounted onto an aluminium stub using conducting double-sided adhesive tape. A thin layer of gold was applied using a sputter coater (20 mA, 120 s).

### Gravimetric Vapour Sorption (GVS)

Sorption isotherms were obtained using a SMS DVS Intrinsic moisture sorption analyser, controlled by DVS Intrinsic Control software. The sample temperature was maintained at 25 °C by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of % RH was constantly monitored by a microbalance (accuracy±0.005 mg).

Typically, 5-30 mg of sample was placed in a tared mesh stainless steel basket under ambient conditions. The sample was loaded and unloaded at 40% RH and 25 °C (typical room conditions). A moisture sorption isotherm was performed as outlined below (2 scans per complete cycle). The standard isotherm was performed at 25 °C at 10% RH intervals over a 0-90% RH range. Typically, a double cycle (4 scans) was carried out. Data analysis was carried out within Microsoft Excel using the DVS Analysis Suite.

| Parameter | Value |
|---|---|
| Adsorption - Scan 1 | 40-90 |
| Desorption, Adsorption - Scan 2 | 90-0, 0-40 |
| Intervals (% RH) | 10 |
| Number of Scans | 4 |
| Flow rate (ml/min) | 200 |
| Temperature (°C) | 25 |
| Stability (°C/min) | 0.2 |
| Sorption Time (hours) | 6 hour time out |
| Number of cycles | 2 |

The sample was recovered after completion of the isotherm and re-analysed by XRPD.

### Chemical Purity Determination by High-Performance Liquid Chromatography (HPLC)

Purity analysis was performed on an Agilent HP1100/Infinity II 1260 series system equipped with a diode array detector and using OpenLAB software. The full method details are provided below:

HPLC Method No. 1

| Parameter | Value | | |
|---|---|---|---|
| Sample Preparation | 0.4 mg/ml in acetonitrile:water 1:1 | | |
| Column | Supelco Ascentis Express C18 2.7 μm | | |
| | 100 × 4.6 mm | | |
| Column Temperature (°C) | 25 | | |
| Injection (μl) | 5 | | |
| Detection: | 255, 90 | | |
| Wavelength, Bandwidth (nm) | | | |
| Flow Rate (ml/min) | 2 | | |
| Phase A | 0.1% TFA in water | | |
| Phase B | 0.085% TFA in acetonitrile | | |
| | Time (min) | % Phase A | % Phase B |
| Timetable | 0 | 95 | 5 |
| | 6 | 5 | 95 |
| | 6.2 | 95 | 5 |
| | 8 | 95 | 5 |

HPLC Method No. 2

| Parameters | Value | | |
|---|---|---|---|
| Test sample make-up: | 0.5 mg/mL | | |
| Column: | XBridge C18, 150 × 4.6 mm, 3.5 μm | | |
| Pharmorphix column no: | 303 | | |
| Column Temperature (°C): | 35 | Autosampler | RT (not |
| | | temperature | specified in the |
| | | (°C) | method) |
| Injection (μl): | 5 μl | | |
| Detection: Wavelength, | 220 nm | | |
| Bandwidth(nm): | 4 nm | | |
| Flow Rate (ml · min − 1): | 1 ml/min | | |
| Phase A: | 0.05% TFA in H2O | | |
| Phase B: | 0.05% TFA in can | | |
| | Time (min) | % Phase A | % Phase B |
| Timetable: | 0.0 | 90 | 10 |
| | 12.0 | 10 | 90 |
| | 15.0 | 10 | 90 |
| | 15.1 | 90 | 10 |
| | 20.0 | 90 | 10 |

Unless stated within the appropriate data tables, all HPLC purity data reported has been run using the HPLC Method No. 2.

### Single Crystal X-Ray Diffraction (SCXRD)

Data were collected on a Rigaku Oxford Diffraction XtaLAB Synergy-S diffractometer equipped with a dualflex source (Cu at Zero), HyPix-6000HE detector and an Oxford Cryosystems Cobra cooling device. The data were collected using Cu Kα radiation as stated in the experimental tables. Structures were solved and refined using the Shelx suite of programs and OLEX was used as an interface to view the structures with and produce figures. Unless otherwise stated, hydrogen atoms attached to carbon were placed geometrically and allowed to refine with a riding isotropic displacement parameter. A reference diffractogram for the crystal structure was generated using Mercury.

### Example 1. Exemplary Preparation of Form 1

Compound A was dissolved in IPA (8.6×) and the temperature raised to 50 °C. Seeds (0.01×) were added to the reaction at 40 °C. The reaction kept at 40 °C for 2 hours then 30 °C for 2 hours. n-Heptane slowly was added (21×) and the mixture stirred a further 9 hours at 30 °C. The reaction was then cooled (5 °C) and filtered. The cake was washed with n-Heptane to yield Form 1 (90% yield).

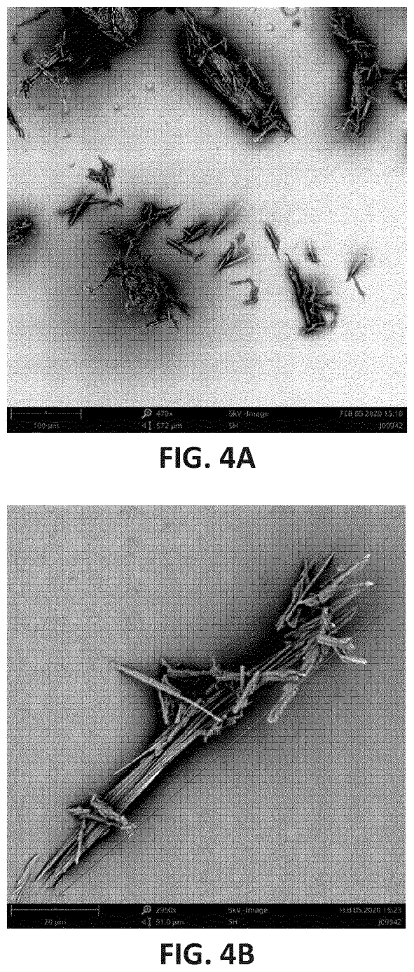

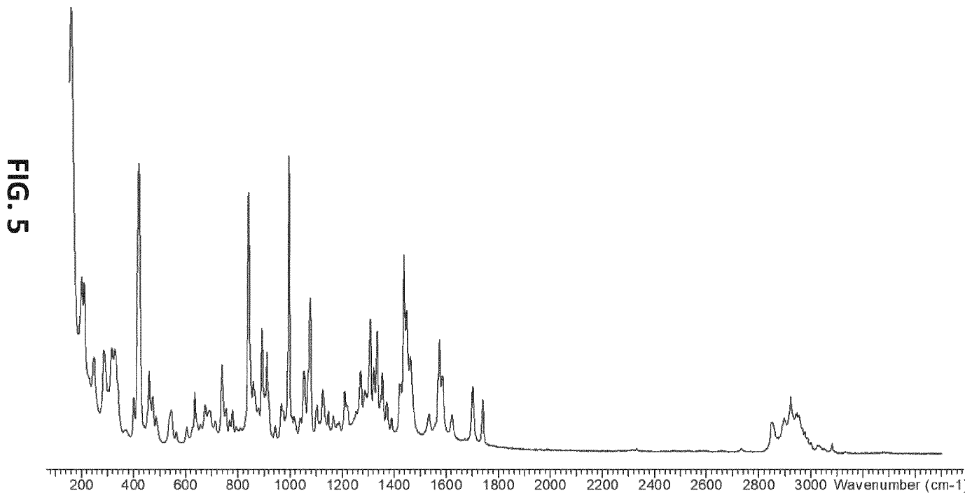

Form 1 was characterized using a wide range of techniques to investigate the solid form and chemical properties (FIGS. 1-6B). A summary of the results is shown below:

TABLE A
Form 1

| XRPD | | Crystalline, Form 1 |
|---|---|---|
| HPLC Purity | | 99.2% |
| (Method No. 1) | | |
| HPLC Purity | | 99.4% |
| (Method No. 2) | | |
| ¹H-NMR | | Collected in d₆-DMSO, 27 protons assigned |
| | | confirming structure |
| TGA | | Degradation ~220 °C |
| DSC | | Sharp endotherm onset ~128.7 °C (97 J/g) |
| PLM/SEM | | Needles various sizes ~150 μm |
| Raman | | Collected as reference, Raman active |
| GVS | | Non-hygroscopic |
| | | <0.1% water uptake between 0-90% RH |
| XRPD Post GVS | | Form 1 |
| Scanning IC | | No anion or cation |
| Solubility | FeSSIF | 0.014 mg/ml |
| | FaSSIF | 0.026 mg/ml |
| | SGF | 0.0047 mg/ml |
| | pH = 1.2 | <0.01 mg/ml |
| | pH = 5 | <0.01 mg/ml |
| | pH = 6.5 | <0.01 mg/ml |
| | pH = 7.4 | <0.01 mg/ml |
| | pH = 8.5 | <0.01 mg/ml |
| Storage | 25 °C/97% RH | Form 1, 99.4% HPLC (Method No. 1) |
| at | 40 °C/75% RH | Form 1, 98.8% HPLC (Method No. 1) |

Form 1 is anhydrous and thermally stable as shown by thermal analysis. TGA showed no weight loss before degradation, which occurred from ~220 °C and DSC showed a melt endotherm onset at 128.7 °C Form 1 is non-hygroscopic, GVS showed an increase of <0.1% between 40-90% RH and no hysteresis and remained as Form 1. The sample was 99.4% pure by HPLC and ion chromatography showed no residual cations/anions were present in the sample. The particles in the supplied batch were of needle morphology of various sizes with an average ~150 μm. The Raman spectrum was collected as reference showing multiple signals. The scanning IC showed no trace of cation or anions present.

### Example 2. Exemplary Preparation of Form 2

Form 1 (500 mg) was dissolved in 30 ml 2-methyl-1-propanol at 5 °C. Heptane (30 ml) was added and stirred for 10 minutes, after additional heptane was added (30 ml) and stirred overnight giving a white precipitate. The suspension was filtered under vacuum and dried for 30 minutes. XRPD analysis showed this to be Form 1. The mother liqueurs were concentrated by evaporation and seeded with Form 2 then evaporated to dryness. Yield: 63.9%.

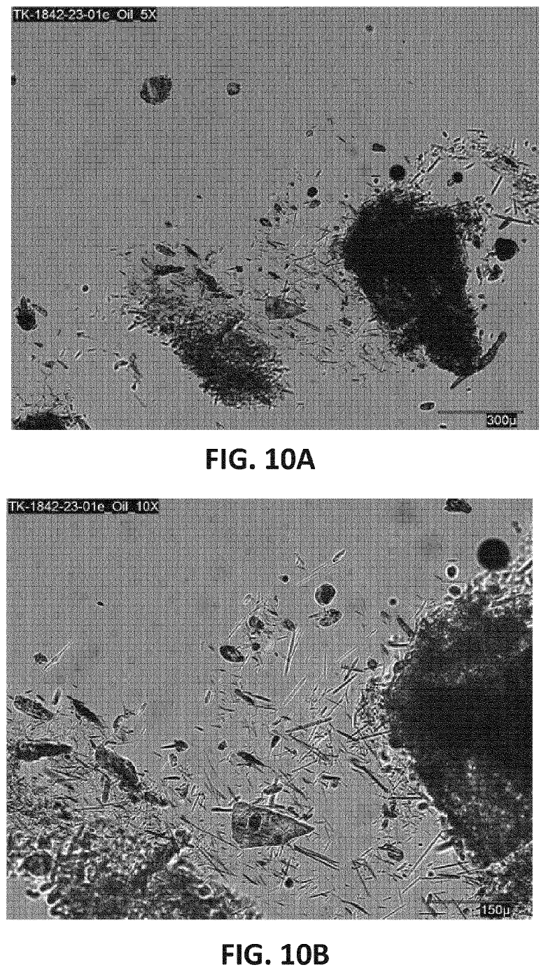

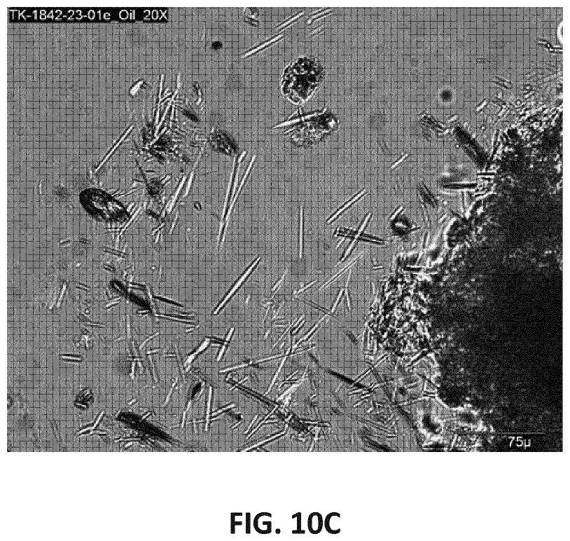

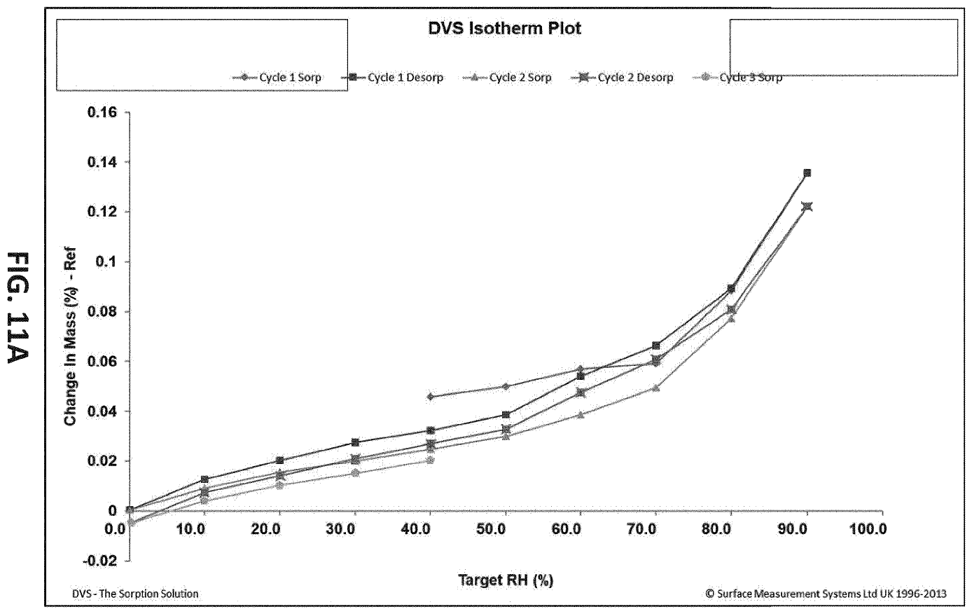

Form 2 was characterized using a wide range of techniques to investigate the solid form and chemical properties (FIGS. 7-11B). A summary of the results is shown below. Table B and Table C showed results of characterization of two batches of Form 2.

TABLE B
Form 2

| XRPD | | Crystalline, Form 2 |
|---|---|---|
| HPLC | | 99.2% |
| (Method No. 1) | | |
| ¹H-NMR | | Collected in d₆-DMSO, 27 protons |
| | | assigned confirming structure |
| TGA | | Degradation ~200 °C |
| DSC | | Sharp endotherm onset ~129.3 °C (89 J/g) |
| Storage at | 25 °C/97% RH | Crystalline - Form 2 |
| | 40 °C/75% RH | Crystalline - Form 2 |

Analysis showed crystalline Form 2 by XRPD, and high purity by HPLC (99.2%). ¹H-NMR analysis matched the expected structure and thermal analysis showed no weight loss before degradation by TGA, and a sharp endotherm onset ~129.3 °C (89 J/g). Form 2 was found to be stable at elevated storage at 25 °C/97% RH and 40 °C/75% RH for 1 week.

TABLE C
Form 2

| XRPD | | Crystalline - Form 2 |
|---|---|---|
| HPLC Purity | | 96.2% |
| (Method No. 1) | | |
| ¹H-NMR | | Consistent with supplied structure |
| TGA | | No weight loss before degradation ~190 °C |
| DSC | | Sharp endotherm onset 128.4 °C (89 J/g) |
| PLM | | Needles ~50-75 μm |
| GVS | | Non-hygroscopic |
| | | ~0.15% reversible uptake from 0-90% RH |
| | | 0.09% uptake 40-90% RH, no hysteresis |
| | | observed |
| XRPD Post GVS | | Crystalline - Form 2 |
| Storage at | 25 °C/97% RH | Crystalline - Form 2/HPLC 96.2% |
| | | (Method No. 1) |
| | 40 °C/75% RH | Crystalline - Form 2/HPLC 97.9% |
| | | (Method No. 1) |

Analysis by XRPD showed Form 2, and the 1H-NMR matched the expected structure. Thermal analysis showed no weight loss before degradation which started from ~190 °C and no thermal events were observed before a large sharp endotherm with an onset temperature at ~128.4 °C (89.4° J/g) by DSC. Microscopy showed the particles to be needles between 50-75 μm. Form 2 was found to be non-hygroscopic with no hysteresis by GVS and remained Form 2 by XRPD after the GVS experiment. The solid form was also found to be stable at elevated conditions.

### Example 3. Solvent and Solubility Study of Morphic Forms

Solubility of Form 1 was measured in several single solvents using Form 2 as starting material, respectively. Results showed Form 2 converted to Form 1 in all selected solvents except for water after slurry at 20-25 °C. Form 1 exhibited good solubility in MeOH, Acetone, MEK, EtOAc and IPAc, moderate solubility in EtOH and IPA, and poor solubility in n-heptane and water. Thus, MeOH, Acetone, MEK, EtOAc and IPAc were selected for further chemical stability evaluation before starting solubility test with anti-solvents.

TABLE D

| Starting | | | Solubility | |
|---|---|---|---|---|
| Material | Solvent | T/°C | (mg/mL) | XRPD |
| Form 2 | MeOH | 20-25 | 77 | Form 1 |
| | EtOH | | 45 | |
| | IPA | | 24 | |
| | Acetone | | 167 | |
| | MEK | | 175 | |
| | EtOAc | | 98 | |
| | IPAc | | 48 | |
| | n-heptane | | 3 | |
| | H₂O | | 1 | Form 2 |

Solubility of Form 1 was further measured in IPAc/Heptane system with different ratio at 0-50 °C. Solubility data showed that a cooling and anti-solvent crystallization is an available design in this solvent system.

TABLE E

| | | Ratio | | Solubility | |
|---|---|---|---|---|---|
| Starting material | Solvent | (v/v) | T/°C | (mg/mL) | XRPD |
| Form 1 | IPAc/Heptane | 1/0 | 50 | 170.6 | Form 1 |
| | | 9/1 | | 120.0 | |
| | | 4/1 | | 75.2 | |
| | | 3/1 | | 65.2 | |
| | | 2/1 | | 53.4 | |
| | | 1/1 | | 34.4 | |
| | | 1/2 | | 18.0 | |
| | | 1/3 | | 8.0 | |
| | | 1/0 | 40 | 80.2 | Form 1 |
| | | 1/0 | 30 | 59.5 | |
| | | 2/1 | | 27.7 | |
| | | 1/1 | | 12.1 | |
| | | 1/2 | | 4.8 | |
| | | 1/3 | | 2.9 | |
| | | 1/1 | 0-5 | 6.4 | Form 1 |
| | | 1/2 | | 3.4 | |
| | | 1/3 | | 1.7 | |

### Example 4. Chemical Stability Evaluation of Morphic Forms

Chemical stability of this compound was evaluated in MeOH, Acetone, MEK, EtOAc, and IPAc at different temperature points. The table below showed that the compound was chemical stable in IPAc at 50 °C for 24 h. However, serious degradation was observed in MeOH and MEK and slight degradation occurred in Acetone and EtOAc after stirred for 23 h.

TABLE F

| Starting | | | Time/h | | |
|---|---|---|---|---|---|
| Material | Solvent | T/°C | 0 h | 5-6 h | 23-24 h |
| Compound A | MeOH | 50 | 99.12% | 98.03% | 90.58% |
| | Acetone | 40 | 99.57% | 99.55% | 98.06% |
| | MEK | 40 | 99.52% | 99.51% | 93.28% |
| | | 50 | 99.38% | 92.75% | 87.20% |
| | EtOAc | 40 | 99.49% | 99.46% | 99.46% |
| | | 50 | 99.43% | 99.31% | 99.27% |
| | IPAc | 50 | 99.70% | 99.73% | 99.76% |
| | | | | (5 h) | (24 h) |

### Example 5. Crystallization Study of Morphic Forms

Crystallization process in IPAc/n-heptane was conducted based on the solubility data. One batch experiment was carried out using 8 vol IPAc for dissolution at 50 °C. Then, 2 vol heptane was charged to the ratio of 4/1 at 50 °C for producing supersaturation. After adding 1% seed and aging for 3 h, 22 vol heptane was dosed into the system in 8 h at 50 °C. Then, the suspension was cooled down to 0-5 °C in 6 h. This process gave correct form with good purity, acceptable mother liquor loss and low residual solvent. However, some solid was observed sticking on the wall, which and might produce a lower solid yield. This may be due to the evaporation of solvent, leading to precipitation of solid on the wall during anti-solvent addition at high temperature.

Another batch crystallization experiment was performed at lower temperature to avoid solid-sticking. After dissolution in 8 vol IPAc at 50 °C, the solution was cooled down to 40 °C for seeding. Then the contents was further cooled down to 30 °C for anti-solvent addition. The reactor was clean and no solid-sticking phenomenon. This optimized process gave good results as before.

A 20 g scale-up experiment was carried out followed with same process. Finally, scale-up experiment generated 96.5% solid yield, correct form, and hundreds ppm of residual solvent. Results showed this process was scaled-up successfully.

TABLE G

| | | | Anti- | | |
|---|---|---|---|---|---|
| S.M. | Dissolution | Seeding | Solvent | Cooling | Results |
| 1 g | 8 v IPAc at | 1% Form 1 | Charge | Cool to | XRPD: Form 1 |
| (Purity: | 50 °C | in IPAc/Heptane | Heptane | 0-5 °C | Solid sticking observed. |
| 99.67%) | | 4/1 at 50 °C | to 1/3 in | in 6 h | Loss in ML: 6%; |
| | | | 8 h at 50 | | Purity: 99.7% |
| | | | °C | | Assay: 99.8% |
| | | | | | RS of IPAc: 100 ppm; |
| | | | | | RS of Heptane: 100 ppm; |
| 1 g | 8 v IPAc at | 1% Form 1 | Charge | Cool to | XRPD: Form 1 |
| (Purity: | 50 °C | in IPAc at | Heptane | 0-5 °C | Loss in ML: 6%; |
| 99.67%) | | 40 °C | to 1/3 in | in 4 h | Purity: 99.7% |
| | | | 8 h at 30 | | Assay: 99.3%; |
| | | | °C | | RS of IPAc: 100 ppm; |
| | | | | | RS of Heptane: <100 ppm; |
| 20 g | 8 v IPAc at | 1% Form 1 | Charge | Cool to | Yield by solid: 96.5%; |
| (Purity: | 50 °C | in IPAc at | Heptane | 0-5 °C | ML Loss: 3.6%; |
| 98.73%; | | 40 °C | to 1/3 in | in 2 h | XRPD: Form 1 |
| Assay: | | | 7 h at 30 | | Purity: 99.0% |
| 92.1%) | | | °C | | Assay: 98.4% |
| | | | | | RS of IPAc: 337 ppm |
| | | | | | RS of heptane: <100 ppm |
| | | | | | ROI: 0.02%; KF: 0.19% |

### Example 6. Drying Stability Test of Morphic Forms

Drying stability was evaluated at 50 °C under vacuum for total 70 h, results exhibited that no degradation and form change during drying. Compound A (Form 1) is demonstrated to be chemically and physically stable at elevated temperatures.

TABLE I

| Drying condition | | Results | | |
|---|---|---|---|---|
| Temperature | Time | Purity | R.S. | XRPD |
| 50 °C | 0 h | 99.0% | / | Form 1 |
| | 22 h | 99.1% | IPAc: 300 ppm | Form 1 |
| | | | Heptane: 100 ppm | |
| | 70 h | 99.1% | IPAc: 200 ppm | Form 1 |
| | | | Heptane: 100 ppm | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms may include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A morphic form of Compound A:

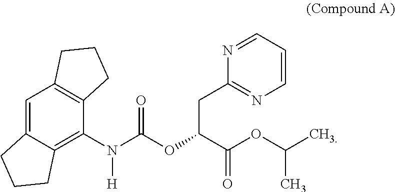 [SMILES: CC(C)OC(=O)[C@@H](Cc1ncccn1)OC(=O)Nc1c2c(cc3c1CCC3)CCC2]

2. The morphic form of claim 1, being a crystalline form of Compound A.

3. The morphic form of any one of the preceding claims, being Form 1 of Compound A.

4. The morphic form of any one of the preceding claims, wherein the morphic form is characterized by at least one of the following characteristics:
(a) an XRPD pattern comprising signals at 8.7±0.5, 15.3±0.5, and 15.9±0.5° 2θ using Cu Kα radiation;
(b) an XRPD pattern comprising signals at 7.9±0.5, 8.7±0.5, 15.3±0.5, and 15.9±0.5° 2θ using Cu Kα radiation;
(c) an XRPD pattern comprising signals at 7.9±0.5, 8.7±0.5, 15.3±0.5, 15.9±0.5, and 24.4±0.5° 2θ using Cu Kα radiation;
(d) an XRPD pattern comprising signals at 7.9±0.5, 8.7±0.5, 15.3±0.5, 15.9±0.5, 22.3±0.5, and 24.4±0.5° 2θ using Cu Kα radiation;
(e) an XRPD pattern comprising signals at 7.9±0.5, 8.7±0.5, 15.3±0.5, 15.9±0.5, 22.3±0.5, 23.9±0.5, and 24.4±0.5° 2θ using Cu Kα radiation; or
(f) an XRPD pattern comprising signals at 5.9±0.5, 7.9±0.5, 8.7±0.5, 15.3±0.5, 15.9±0.5, 22.3±0.5, 23.9±0.5, and 24.4±0.5° 2θ using Cu Kα radiation.

5. The morphic form of any one of the preceding claims, wherein the morphic form is characterized by an XRPD pattern comprising one or more signals as described in Table 1.

6. The morphic form of any one of the preceding claims, wherein the morphic form is characterized by an XRPD pattern substantially similar to that set forth in FIG. 2.

7. The morphic form of claim 1 or claim 2, being Form 2 of Compound A.

8. The morphic form of any one of claims 1-2 and 7, wherein the morphic form is characterized by at least one of the following characteristics:
(a) an XRPD pattern comprising signals at 7.6±0.5, 9.4±0.5, and 15.9±0.5° 2θ using Cu Kα radiation;
(b) an XRPD pattern comprising signals at 7.6±0.5, 9.4±0.5, 15.2±0.5, and 15.9±0.5° 2θ using Cu Kα radiation;
(c) an XRPD pattern comprising signals at 6.0±0.5, 7.6±0.5, 9.4±0.5, 15.2±0.5, and 15.9±0.5° 2θ using Cu Kα radiation;
(d) an XRPD pattern comprising signals at 6.0±0.5, 7.6±0.5, 9.4±0.5, 15.2±0.5, 15.9±0.5, and 21.5±0.5° 2θ using Cu Kα radiation;
(e) an XRPD pattern comprising signals at 6.0±0.5, 7.6±0.5, 9.4±0.5, 15.2±0.5, 15.9±0.5, 21.5±0.5, and 23.9±0.5° 2θ using Cu Kα radiation; and
(f) an XRPD pattern comprising signals at 6.0±0.5, 7.6±0.5, 9.4±0.5, 15.2±0.5, 15.9±0.5, 21.2±0.5, 21.5±0.5, and 23.9±0.5° 2θ using Cu Kα radiation.

9. The morphic form of any one of claims 1-2 and 7-8, wherein the morphic form is characterized by an XRPD pattern comprising one or more signals as described in Table 2.

10. The morphic form of any one of claims 1-2 and 7-9, wherein the morphic form is characterized by an XRPD pattern substantially similar to that set forth in FIG. 8.

11. The morphic form of any one of the preceding claims, having a purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

12. The morphic form of any one of the preceding claims, having a morphic purity of about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater.

13. The morphic form of any one of the preceding claims, exhibiting less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 25 °C and 97% relative humidity (RH), over 1 day, 2 days, 3 days, 4 days, 5 days, 6 days, one week, two weeks, three weeks, one month, two months, three months, or four months.

14. The morphic form of any one of the preceding claims, exhibiting less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity, under 40 °C and 75% relative humidity (RH), over 1 day, 2 days, 3 days, 4 days, 5 days, 6 days, one week, two weeks, three weeks, one month, two months, three months, or four months.

15. A method of preparing a morphic form of Compound A of any one of the preceding claims.

16. A pharmaceutical composition comprising a morphic form of Compound A of any one of the preceding claims, and one or more pharmaceutically acceptable carrier or excipient.

17. A method of preventing or treating a disease or disorder in a subject, comprising administering to the subject the morphic form of Compound A of any one of claims 1-14 or the pharmaceutical composition of claim 16.

18. The morphic form of Compound A of any one of claims 1-14 or the pharmaceutical composition of claim 16 for use in preventing or treating a disease or disorder in a subject.

19. A method of inhibiting inflammasome activity in a subject, comprising contacting a cell with the morphic form of Compound A of any one of claims 1-14 or the pharmaceutical composition of claim 16.

20. The method, morphic form, pharmaceutical composition, or use of any one of claims 17-19, wherein the subject is human.
